(19) United States
(12) Patent Application Publication
(10) Pub. No.: US 2016/0273042 A1
(43) Pub. Date: Sep. 22, 2016

(54) GENETIC MARKERS OF ANTIPSYCHOTIC RESPONSE

(71) Applicant: SUREGENE, LLC, Louisville, KY (US)

(72) Inventors: Timothy RAMSEY, Shelbyville, KY (US); Mark BRENNAN, Jeffersonville, IN (US)

(73) Assignee: Clinical Reference Laboratory, Inc., Lenexa, KS (US)

(21) Appl. No.: 15/034,200

(22) PCT Filed: Nov. 6, 2014
(86) PCT No.: PCT/US14/64235

§ 371 Date: May 4, 2016

Related U.S. Application Data
(60) Provisional application No. 61/901,238, filed on Nov. 7, 2013.

(51) Int. Cl. C12Q 1/68; A61K 31/496; A61K 31/519; A61K 31/5415; A61K 31/554; A61K 31/5513; A61K 45/06

(52) U.S. Cl. CPC C12Q 1/6883; A61K 31/5513; A61K 31/496; A61K 45/06; A61K 31/5415; A61K 31/554; A61K 31/519; C12Q 2600/156; C12Q 2600/106; C12Q 2600/158; C12Q 2600/118

(57) ABSTRACT

Provided herein are genetic markers for predicting response to antipsychotic treatment. Genotyping of the disclosed SNPs can be used to predict response to antipsychotic drugs in patients suffering from schizophrenia. There is provided method of treating a subject with an antipsychotic medication comprising obtaining genetic information from the subject comprising the sequence of SNPs rs6923761, rs2300615, and rs1042044 to provide the GLP1 R Antipsychotic Response Phenotype (GARP) genetic signature of the subject; and administering an antipsychotic medication to the subject based on the GARP genetic signature of the subject.

This application claims benefit of priority to U.S. Provisional Application Ser. No. 61/901,238, filed Nov. 7, 2013, the entire contents of which are hereby incorporated by reference.

This invention was made with government support under Grant No. MH078437 awarded by the National Institutes of Health. The government has certain rights in the invention.

### BACKGROUND OF THE INVENTION

1. Field of the Invention

The present invention relates generally to the fields of medicine, genetics, and psychiatry. More particularly, it concerns genetic markers that are associated with response to antipsychotic treatments.

2. Description of Related Art

Numerous drugs exist to treat psychotic disorders, such as schizophrenia (SZ), related SZ-spectrum disorders (including schizotypal personality disorder (SPD) and schizoaffective disorder (SD)), and bipolar disorders (BD). Most of these drugs fall into one of two categories, typical (first generation) and atypical (second generation).

Although head to head studies of large groups of patients, either in the acute phase or outpatient treatment, show that most atypical antipsychotic drugs are equally efficacious for positive symptoms, there are individual differences in response to specific drugs based on differences in drug pharmacology and metabolism, combined with genetic differences between patients. There are currently no proven ways to identify which antipsychotic drug is optimal for a given patient. Thus, patients switch from one drug to another when response is not considered to be adequate or side effects are intolerable. This switching of medication incurs a variety of increased costs, both economic and patient and caregiver hardship. Moreover, the limited or partial response that is often seen with antipsychotics leads to polypharmacy, where physicians prescribe two or more antipsychotic drugs plus mood stabilizers and/or antidepressants. Polypharmacy increases medication costs and significantly increases the likelihood of adverse advents and drug interactions (Stahl and Grady, 2006). On average, each patient may change medications three times for finding one that works. Additionally, the current drugs have significant side-effects. This combination of side-effects and limited efficacy create a vast unmet need for selecting the optimal antipsychotic for each patient.

Pharmacogenomics, using genetic variation to predict altered response and side-effects profiles, will be important for enhanced patient care going forward. There continues to exist, therefore, a need to identify specific genetic variations that are associated with psychotic disorders such as schizophrenia.

### SUMMARY OF THE INVENTION

In a first embodiment the invention provides new genetic markers that can be used to guide therapeutic intervention with antipsychotic medications. It has been determined that certain polymorphisms (e.g., single nucleotide polymorphisms (SNPs)) in the glucagon-like peptide 1 receptor gene (GLP1R) and linked genetic elements can be used to predict response to antipsychotic medications. By assessing the presence (or absence) of these polymorphism therapeutic intervention can be optimized to maximize efficacy, while reducing negative side effects.

Thus, in a first embodiment, there is provided method of treating a subject with an antipsychotic medication comprising obtaining genetic information from the subject comprising the sequence of SNPs rs6923761, rs2300615, and rs1042044 to provide the GLP1R Antipsychotic Response Phenotype (GARP) genetic signature of the subject; and administering an antipsychotic medication to the subject based on the GARP genetic signature of the subject. In some aspects, obtaining genetic information to provide a GARP genetic signature can comprise determining the sequence at the nucleotide positions of SNPs rs6923761, rs2300615, and rs1042044. In still further aspects, obtaining genetic information to provide a GARP genetic signature can comprise determining the sequence at SNPs in linkage disequilibrium with SNP positions rs6923761, rs2300615, and rs1042044. For example, a GARP genetic signature can be provided by determining the sequence at SNP positions rs9296283 rs7766275 rs2300615 and rs1042044. In further aspects, a GARP genetic signature can be provided by determining the sequence at SNP positions rs10305439 rs742764 rs2268650 rs910170 rs6923761 rs9296283 rs7766275 rs2300615 rs2235868 and rs1042044. In accordance with certain in embodiments, determining the sequence can comprise consulting chart or an electronic medium comprising a subjects genetic sequence (or a part thereof). In further aspects, determining the sequence can comprise obtaining a sample from a subject and sequencing genetic material in the sample.

In one aspect a GARP genetic signature of the embodiments is a GARP-1 genetic signature corresponding to SNPs rs6923761 (G), rs2300615 (T), and rs1042044 (A) on both chromosomes. Thus, a subject with a GARP-1 genetic signature has (a) rs6923761 (G), rs2300615 (T), and rs1042044 (A) haplotype and (b) a rs6923761 (G), rs2300615 (T), and rs1042044 (A) haplotype.

Accordingly, in one aspect a method of the embodiments comprises selecting a subject having (e.g., known to have) a GARP-1 genetic signature corresponding to SNPs rs6923761 (G), rs2300615 (T), and rs1042044 (A) on both chromosomes and administering first antipsychotic medication selected from the group consisting of olanzapine, risperidone, and clozapine to the selected subject. In some aspects, a method further comprises determining the efficacy or the side effects associated with administering the first antipsychotic medication and/or administering a second antipsychotic medication to the subject, (e.g., selected from the group consisting of olanzapine, risperidone, and clozapine), if the first antipsychotic medication is determined to have insufficient efficacy or unacceptable side effects. In further aspects, a subject having a GARP-1 genetic signature is not administered ziprasidone, perphenazine or aripiprazole. In still further aspects, a subject having a GARP-1 genetic signature is treated with a GLP1R agonist in conjunction with an antipsychotic medication, such as olanzapine or clozapine.

In a further embodiment a method comprises selecting a subject having a GARP-1 genetic signature corresponding to SNPs rs6923761 (G), rs2300615 (T), and rs1042044 (A) on both chromosomes and administering an antipsychotic medication wherein the antipsychotic medication is not ziprasidone, perphenazine or aripiprazole.

In yet a further embodiment a method is provided for using the antipsychotic aripiprazole comprising determining whether a subject has the GARP-1 genetic signature corresponding to SNPs rs6923761 (G), rs2300615 (T), and rs1042044 (A) on both chromosomes; and treating the subject with aripiprazole if the subject is GARP-1 negative or treating the subject with a non-aripiprazole antipsychotic if the subject is GARP-1 positive.

In still a further embodiment a method of using olanzapine is provided comprising, determining whether a subject has the GARP-1 genetic signature corresponding to SNPs rs6923761 (G), rs2300615 (T), and rs1042044 (A) on both chromosomes; and treating the subject with olanzapine if the subject is GARP-1 positive or treating the subject with a non-olanzapine antipsychotic if the subject is GARP-1 negative.

In still a further embodiment a method of using the antipsychotic ziprasidone is provided, comprising determining whether a subject has the GARP-1 genetic signature corresponding to SNPs rs6923761 (G), rs2300615 (T), and rs1042044 (A) on both chromosomes; and treating the subject with ziprasidone if the subject is GARP-1 negative or treating the subject with a non-ziprasidone antipsychotic if the subject is GARP-1 positive

In yet a further embodiment there is provided a method of treating a subject comprising selecting a subject having (e.g., known to have) a GARP-1 genetic signature corresponding to SNPs rs6923761 (G), rs2300615 (T), and rs1042044 (A) on both chromosomes; and administering (i) a first antipsychotic medication selected from the group consisting of olanzapine and clozapine; and (ii) a GLP1R agonist to the selected subject. For example, the GLP1R agonist and the first antipsychotic medication are administered separately or in the same composition. Thus, the GLP1R agonist may be administered before, after or essentially simultaneously with the antipsychotic medication selected. In certain aspects, the GLP1R agonist and/or the antipsychotic medication is administered orally or by injection. Examples of GLP1R agonists that may be used according to the embodiments include, without limitation, exenatide and liraglutide.

In a further aspect a GARP genetic signature of the embodiments is a GARP-2 genetic signature corresponding to (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and rs1042044 (C); or (iv) SNPs rs6923761 (G), rs2300615 (T), and rs1042044 (C) on a second chromosome. Thus, a subject with a GARP-2 genetic signature has (a) SNPs rs6923761 (A), rs2300615 (T), and rs1042044 (C), and rs1042044 (A) haplotype and (b) a haplotype selected from (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and rs1042044 (C); or (iv) SNPs rs6923761 (G), rs2300615 (T), and rs1042044 (C).

Thus, in one embodiment a method comprises selecting a subject having (e.g., known to have) a GARP-2 genetic signature corresponding to: (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and rs1042044 (C); or (iv) SNPs rs6923761 (G), rs2300615 (T), and rs1042044 (C) on a second chromosome; and administering a first antipsychotic medication selected from the group consisting of perphenazine, risperidone and ziprasidone to the selected subject. In some aspects the method further comprises determining the efficacy or the side effects associated with administering the first antipsychotic medication and/or administering a second antipsychotic medication to the subject, (e.g., selected from the group consisting of perphenazine, risperidone and ziprasidone), if the first antipsychotic medication is determined to have insufficient efficacy or unacceptable side effects. In further aspects, the selected subject (having a GARP-2 genetic signature) is not administered clozapine, olanzapine, or quetiapine.

In a further embodiment a method comprises selecting a subject having a GARP-2 genetic signature corresponding to: (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and administering an antipsychotic medication to the subject, wherein the antipsychotic medication is not clozapine, olanzapine, or quetiapine.

In still a further embodiment a method is provided for using the antipsychotic clozapine comprising, determining whether a subject has the GARP-2 genetic signature corresponding to: (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and treating the subject with clozapine if the subject is GARP-2 negative or treating the subject with a non-clozapine antipsychotic if the subject is GARP-2 positive.

In yet a further embodiment a method is provided for using the antipsychotic perphenazine, the method comprising determining whether a subject has the GARP-2 genetic signature corresponding to: (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and treating the subject with perphenazine if the subject is GARP-2 positive or treating the subject with a non-perphenazine antipsychotic if the subject is GARP-2 negative.

In still a further embodiment a method for using the antipsychotic quetiapine is provided comprising, determining whether a subject has the GARP-2 genetic signature corresponding to (a) SNPs rs6923761 (A), rs2300615 (T), and rs1042044 (C) on one chromosome; and (b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and treating the subject with quetiapine if the subject is GARP-2 negative or treating the subject with a non-quetiapine antipsychotic if the subject is GARP-2 positive.

In a further aspect a GARP genetic signature of the embodiments is a GARP-3 genetic signature corresponding to (a) SNPs rs6923761 (G), rs2300615 (G), and rs1042044 (C) on one chromosome; and (b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome. Thus, a subject with a GARP-3 genetic signature has (a) SNPs rs6923761 (G), rs2300615 (G), and rs1042044 (C) haplotype and (b) a haplotype selected from (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C).

Thus, in one embodiment a method comprises selecting a subject having (e.g., known to have) a GARP-3 genetic signature corresponding to (a) SNPs rs6923761 (G), rs2300615 (G), and rs1042044 (C) on one chromosome; and (b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and administering first antipsychotic medication to the subject selected from the group consisting of clozapine, quetiapine and ziprasidone to the selected subject. In some aspects, a method further comprises determining the efficacy or the side effects associated with administering the first antipsychotic medication and/or administering a second antipsychotic medication to the subject (e.g., selected from the group consisting of clozapine, quetiapine and ziprasidone), if the first antipsychotic medication is determined to have insufficient efficacy or unacceptable side effects. In some aspects, the subject (having a GARP-3 genetic signature) is not administered olanzapine or risperidone.

In yet a further embodiment a method comprises selecting a subject having a GARP-3 genetic signature corresponding to (a) SNPs rs6923761 (G), rs2300615 (G), and rs1042044 (C) on one chromosome; and (b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and administering an antipsychotic medication to the subject, wherein the antipsychotic medication is not olanzapine or risperidone.

In still a further embodiment a method is provided for using the antipsychotic risperidone, comprising determining whether a subject has the GARP-3 genetic signature corresponding to: (a) SNPs rs6923761 (G), rs2300615 (G), and rs1042044 (C) on one chromosome; and (b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and treating the subject with risperidone if the subject is GARP-3 negative or treating the subject with a non-risperidone antipsychotic if the subject is GARP-3 positive.

Some aspects of the embodiments involve determining whether genetic material of the subject comprises a haplotype or a diplotype of the embodiments and transferring or storing the information. For example, such information, can be recorded and maintained in a tangible medium, such as a computer-readable disk, a solid state memory device, an optical storage device or the like, more specifically, a storage device such as a hard drive, a Compact Disk (CD) drive, a floppy disk drive, a tape drive, a random access memory (RAM), etc. Thus, in certain aspects obtaining a haplotype or a diplotype comprises retrieving information that has been recorded or stored, e.g., in a computer readable medium.

In certain aspects, obtaining haplotype and/or diplotype information involves analyzing the genetic material of the subject to determine the presence or absence of the haplotype. This can be accomplished, for example, by testing the subject's genetic material through the use of a biological sample. In certain embodiments, the methods set forth will thus involve obtaining a biological sample from the subject and testing the biological sample to identify whether an haplotype is present. The biological sample may be any biological material that contains DNA or RNA of the subject, such as a nucleated cell source. Non-limiting examples of cell sources available in clinical practice include hair, skin, nucleated blood cells, buccal cells, any cells present in tissue obtained by biopsy or any other cell collection method. The biological sample may also be obtained from body fluids, including without limitation blood, saliva, sweat, urine, amniotic fluid (the fluid that surrounds a fetus during pregnancy), cerebrospinal fluid, feces, and tissue exudates at the site of infection or inflammation. DNA may be extracted from the biologic sample such as the cell source or body fluid using any of the numerous methods that are standard in the art.

Determining whether the genetic material exhibits a haplotype can be by any method known to those of ordinary skill in the art, such as genotyping (e.g., SNP genotyping) or sequencing. Techniques that may be involved in this determination are well-known to those of ordinary skill in the art. Examples of such techniques include allele specific oligonucleotide hybridization, size analysis, sequencing, hybridization, 5′ nuclease digestion, single-stranded conformation polymorphism analysis, allele specific hybridization, primer specific extension, and oligonucleotide ligation assays. Additional information regarding these techniques is discussed in the specification below.

For haplotype determinations, the sequence of the extracted nucleic acid of the subject may be determined by any means known in the art, including but not limited to direct sequencing, hybridization with allele-specific oligonucleotides, allele-specific PCR, ligase-PCR, HOT cleavage, denaturing gradient gel electrophoresis (DDGE), and single-stranded conformational polymorphism (SSCP) analysis. Direct sequencing may be accomplished by any method, including without limitation chemical sequencing, using the Maxam-Gilbert method, by enzymatic sequencing, using the Sanger method; mass spectrometry sequencing; and sequencing using a chip-based technology. In particular embodiments, DNA from a subject is first subjected to amplification by polymerase chain reaction (PCR) using specific amplification primers. In some embodiments, the method further involves amplification of a nucleic acid from the biological sample. The amplification may or may not involve PCR. In some embodiments, the primers are located on a chip.

Moreover, the inventors contemplate that the genetic structure and sequence, including SNP profiles, of individual subjects will at some point be widely or generally available, or will have been developed by an unrelated third party. In such instances, there will be no need to test or analyze the subject's biological material again. Instead, the genetic information will in such cases be obtained simply by analyzing the sequencing or genotyping outcome of the subject, for example, a SNP profile, a whole or partial genome sequence, etc. These outcomes can then be obtained from or reported by a sequencing or a genotyping service, a laboratory, a scientist, or any genetic test platforms.

A subject as used herein typically refers to a human subject. For example, the subject can have or be at risk for early, intermediate, or aggressive SZ. In some aspects, the subject has one or more risk factors associated with SZ. For instance, the subject may have a relative afflicted with SZ or a genetically-based phenotypic trait associated with risk for SZ. In some aspects, the subject is Caucasian or comprises European ancestry. In further aspects, the subject is African American or comprises African ancestry.

In some further aspects, the method may further comprise reporting the determination to the subject, a health care payer, an attending clinician, a pharmacist, a pharmacy benefits manager, or any person that the determination may be of interest.

Any of the SNPs listed in the Tables herein can be readily mapped on to the publically available human genome sequence (e.g., NCBI Human Genome Build 37.3).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Choosing the correct antipsychotic medication for patients suffering from severe neuropsychiatric illnesses is a major challenge. Fewer than one in three patients suffering from schizophrenia and related disorders will have a robust improvement in symptoms on the first antipsychotic drug prescribed. One out of three patients will be resistant to commonly used drugs. Furthermore, the metabolic side-effects of antipsychotic drugs, most commonly seen with olanzapine and clozapine, result in low compliance, with 50% of patients discontinuing drug use within 6 months of prescription, leading to relapse (return of psychosis) and hospitalization.

Therefore, methods and compositions of the present invention will help to meet this challenge by assisting physicians, patients, lab, or pharmacists with selection or recommendation of appropriate antipsychotic medication. In particular, studies herein demonstrate that certain polymorphisms in GLP1R (or in linked genetic elements) can be used to predict response to antipsychotic medications such as olanzapine, perphenazine, quetiapine, risperidone, and ziprasidone. By assessing the presence (or absence) of these polymorphism therapeutic intervention can be optimized to maximize efficacy, while reducing negative side effects. Moreover, certain of these polymorphisms can be used to predict particular side effects, such as weight gain, which can then be mitigated by further therapeutic intervention. Accordingly, the methods provided here allow antipsychotic therapies to be individualized for the greatest possible effect.

Further embodiments and advantages of the invention are described below.

### I. DEFINITIONS

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

Microsatellites (sometimes referred to as a variable number of tandem repeats or VNTRs) are short segments of DNA that have a repeated sequence, usually about 2 to 5 nucleotides long (e.g., a CA nucleotide pair repeated three times), that tend to occur in non-coding DNA. Changes in the microsatellites sometimes occur during the genetic recombination of sexual reproduction, increasing or decreasing the number of repeats found at an allele, changing the length of the allele. Microsatellite markers are stable, polymorphic, easily analyzed and occur regularly throughout the genome, making them especially suitable for genetic analysis.

“Copy number variation” (CNV), as used herein, refers to variation from the normal diploid condition for a gene or polymorphism. Individual segments of human chromosomes can be deleted or duplicated such that the subject's two chromosomes carry fewer than two copies of the gene or polymorphism (a deletion or deficiency) or two or more copies (a duplication).

“Linkage disequilibrium” (LD) refers to when the observed frequencies of haplotypes in a population does not agree with haplotype frequencies predicted by multiplying together the frequency of individual genetic markers in each haplotype. When SNPs and other variations that comprise a given haplotype are in LD with one another, alleles at the different markers correlate with one another.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing the same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of the hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably, at least partially, single stranded (e.g., denatured) at the time of hybridization. When the target nucleotide sequences are located only in a single region or fraction of a given chromosome, the term “target region” is sometimes used. Targets for hybridization can be derived from specimens that include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al., Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 30% (e.g., at least 40%, 50%, 60%, 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

As used herein, the term “substantially identical” is used to refer to a first nucleotide sequence that contains a sufficient number of identical nucleotides to a second nucleotide sequence such that the first and second nucleotide sequences have similar activities. Nucleotide sequences that are substantially identical are at least 80% (e.g., 85%, 90%, 95%, 97% or more) identical.

The term “nonspecific binding DNA” refers to DNA that is complementary to DNA segments of a probe, which DNA occurs in at least one other position in a genome, outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

As used herein, the term “stratification” refers to the creation of a distinction between subjects on the basis of a characteristic or characteristics of the subjects. Generally, in the context of clinical trials, the distinction is used to distinguish responses or effects in different sets of patients distinguished according to the stratification parameters. In some embodiments, stratification includes distinction of subject groups based on the presence or absence of particular markers or alleles described herein. The stratification can be performed, e.g., in the course of analysis, or can be used in creation of distinct groups or in other ways.

### II. DETERMINATION OF GARP GENETIC SIGNATURE

As detailed above, embodiments of the invention includes methods for determination of the GARP genetic signature in order to select optimal treatments. In particular, GARP is a genetic signature that uses four common haplotypes from the GLP1R gene to determine likely response to various antipsychotic medications. SNPs rs6923761 (A/G), rs2300615 (T/G), and rs1042044 (A/C). Both rs6923761 and rs1042044 SNPs create protein coding changes, serine (A) vs. glycine (G) and leucine (A) vs. phenylalanine (C), respectively. These SNPs describe four common haplotypes, and four difference response phenotypes, three of which (GARP-1, -2 and -3) provide significant information concerning the response phenotype of an individual. These GARP phenotypes as follows:

The GARP-1 genetic signature corresponds to SNPs rs6923761 (G), rs2300615 (T), and rs1042044 (A) on both chromosomes. Thus, a subject with a GARP-1 genetic signature has one rs6923761 (G), rs2300615 (T), and rs1042044 (A) haplotype and one rs6923761 (G), rs2300615 (T), and rs1042044 (A) haplotype.

The GARP-2 genetic signature corresponds to SNPs rs6923761 (A), rs2300615 (T), and rs1042044 (C) on one chromosome and one of four haplotypes on a second chromosome. In particular, the haplotypes on the second chromosome are: (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and rs1042044 (C); or (iv) SNPs rs6923761 (G), rs2300615 (T), and rs1042044 (C).

The GARP-3 genetic signature corresponds to SNPs rs6923761 (G), rs2300615 (G), and rs1042044 (C) on one chromosome and one of three haplotypes on a second chromosome. In particular, the haplotypes on the second chromosome are: (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C).

Using the exemplary SNP markers (i.e., rs6923761, rs2300615, and rs104204) or SNPs in linkage disequilibrium with the exemplary SNPs (see, e.g., Tables 1B and 1C), one can determine the presence or absence of an GARP genetic signature. Using the genetic signature, one can assign subjects to specific categories based on the evaluation of the genetic signature present in the subject and select optimal treatments (atypical antipsychotic, typical antipsychotic, and/or psychosocial intervention) for patients.

Determining a GARP genetic signature can, but need not, include obtaining a sample comprising DNA from a subject, and/or assessing the identity, presence or absence of one or more genetic markers for the GARP genetic signature in the sample. The individual or organization who determines the genetic signature need not actually carry out the physical analysis of a sample from a subject; the genetic signature can include information obtained by analysis of the sample by a third party. Thus the methods can include steps that occur at more than one site. For example, a sample can be obtained from a subject at a first site, such as at a health care provider or at the subject's home in the case of a self-testing kit. The sample can be analyzed at the same or a second site, e.g., at a laboratory, a sequencing or genotyping facility, or other testing facility. Determining a genetic signature can also include or consist of reviewing a subject's medical history or test results, where the medical history or test results includes information regarding the identity, presence or absence of one or more genetic markers in the subject.

Samples that are suitable for use in the methods described herein contain genetic material, e.g., genomic DNA (gDNA). Non-limiting examples of sources of samples include urine, blood, cells, and tissues. The sample itself will typically consist of nucleated cells (e.g., blood or buccal cells), tissue, etc., removed from the subject. The subject can be an adult, a child, a fetus, or an embryo. In some embodiments, the sample is obtained prenatally, either from a fetus or an embryo or from the mother (e.g., from fetal or embryonic cells in the maternal circulation). Methods and reagents are known in the art for obtaining, processing, and/or analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

The sample may be further processed before the detecting step. For example, DNA in a cell or tissue sample can be separated from other components of the sample. The sample can be concentrated and/or purified to isolate DNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., 2003, supra. All samples obtained from a subject, including those subjected to any sort of further processing, are considered to be obtained from the subject.

GARP genetic signature may be determined by any methods known in the art, e.g., gel electrophoresis, capillary electrophoresis, size exclusion chromatography, sequencing, and/or arrays to detect the presence or absence of the marker(s) of the genetic signature. Amplification of nucleic acids, where desirable, can be accomplished using methods known in the art, e.g., PCR.

Methods of nucleic acid analysis to detect polymorphisms and/or polymorphic variants include, e.g., microarray analysis. Hybridization methods, such as Southern analysis, Northern analysis, or in situ hybridizations, can also be used (see Ausubel et al., 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

Other methods include direct manual sequencing (Church and Gilbert, 1988; Sanger et al., 1977; U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989), mobility shift analysis (Orita et al., 1989), restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, for example. See, e.g., U.S. Patent Publication No. 2004/0014095, to Gerber et al., which is incorporated herein by reference in its entirety. In some embodiments, the sequence is determined on both strands of DNA.

In order to detect polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. See e.g., PCR Primer: A Laboratory Manual; McPherson et al., 2000; Mattila et al., 1991; Eckert et al., 1991; and U.S. Pat. No. 4,683,202. Other amplification methods that may be employed include the ligase chain reaction (LCR) (Wu and Wallace, 1989, Landegren et al., 1988), transcription amplification (Kwoh et al., 1989), self-sustained sequence replication (Guatelli et al., 1990), and nucleic acid based sequence amplification (NASBA). Guidelines for selecting primers for PCR amplification are well known in the art. See, e.g., McPherson et al. (2000). A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In one example, a sample (e.g., a sample comprising genomic DNA), is obtained from a subject. The DNA in the sample is then examined to determine a GARP genetic signature as described herein. The genetic signature can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., 1994). The PNA probe can be designed to specifically hybridize to a nucleic acid comprising a polymorphic variant of the GARP genetic signature.

In some embodiments, restriction digest analysis can be used to detect the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Ausubel et al., supra). The digestion pattern of the relevant DNA fragment may indicate the presence or absence of a particular polymorphic variant of the GARP genetic signature and may be therefore indicative of the presence or absence of the GARP genetic signature.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined

Allele-specific oligonucleotides can also be used to detect the presence of a polymorphic variant, e.g., through the use of dot-blot hybridization of amplified oligonucleotides with allele-specific oligonucleotide (ASO) probes (see, for example, Saiki et al., 1986). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism. An allele-specific oligonucleotide probe that is specific for a particular polymorphism can be prepared using standard methods (see Ausubel et al., supra).

Generally, to determine which of multiple polymorphic variants is present in a subject, a sample comprising DNA is obtained from the individual. PCR can be used to amplify a portion encompassing the polymorphic site. DNA containing the amplified portion may be dot-blotted, using standard methods (see Ausubel et al., supra), and the blot contacted with the oligonucleotide probe. The presence of specific hybridization of the probe to the DNA is then detected. Specific hybridization of an allele-specific oligonucleotide probe (specific for a polymorphic variant indicative of susceptibility to altered pharmacological response) to DNA from the subject may determine a GARP genetic signature.

In some embodiments, fluorescence polarization template-directed dye-terminator incorporation (FP-TDI) is used to determine which of multiple polymorphic variants of a polymorphism is present in a subject (Chen et al., 1999). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderborn et al., 2000). Additional methods include, for example, PCR amplification in combination with denaturing high performance liquid chromatography (dHPLC) (Underhill et al., 1997).

The methods can include determining the genotype of a subject with respect to both copies of the polymorphic site present in the genome. For example, the complete genotype may be characterized as −/−, as −/+, or as +/+, where a plus sign indicates the presence of the polymorphic variant of interest, such as rs11960832(T) or rs7975477(T), and a minus sign indicates the absence of the polymorphic variant of interest and/or the presence of the other or wild type sequence at the polymorphic site. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

### III. PSYCHOTIC DISORDERS

Certain aspects of the invention can use GARP genetic signature status to optimize treatments for psychotic disorders, such as schizophrenia (SZ), schizotypal personality disorder (SPD), schizoaffective disorder (SD), and/or bipolar disorders (BD).

Schizophrenia and bipolar disorder are life-long, severely disabling mental illnesses. The clinical criteria for these neuropsychiatric illnesses have continued to evolve through a consensus process organized by the American Psychiatric Association (APA) and published in its Diagnostic and Statistical Manual (DSM) I-IV (American Psychiatric Assoc. Diagostic and Statistical Manual of Mental Disorders, 1994). The inventors disclose here some of the key features of both illnesses as currently conceived, with the full awareness that there is strong evidence for overlap between these disorders in genetic risk factors and response to treatment. Nevertheless, all indications are that DSM-V, which is currently being developed by the APA, will maintain this distinction more or less in the current form. Also because FDA indications for treatment have been and may continue to be given for drugs for each disorder separately, having genetic information which pertains to classification and prediction of response to treatment is of considerable value.

Schizophrenia and bipolar disorder share some common clinical features while differing on others. Schizophrenia is characterized by psychotic symptoms (delusions, hallucinations), disorganized thinking and cognitive impairment and poor social and work function. Additionally, some schizophrenia patients can have severe negative symptoms, including blunted affect and social and emotional withdrawal. Bipolar Disorder is characterized by two main types of mood disturbances, with depression being the most common type and mania, or hypomania less frequent. Psychotic disorders may be present in either the manic or depressive mood phases. Both disorders have a high risk for suicide attempts and completions.

Schizophrenia usually begins in the late teens and early 20's. It affects about 1% of the population. Conversely, bipolar disorder most often occurs in the 3^{rd} and 4^{th} decades of life. Bipolar (BP) Type I affects about 1.5% of the population. BP type II and BP Not Otherwise Specified (NOS) afflict another 2-4% of the population. Life-long drug treatment is often required to minimize the number of acute episodes, the need for hospitalization or assisted living, and to optimize daily functioning. Suicide occurs in 5% of schizophrenia cases and 10% of bipolar disorder cases. Patients with schizophrenia or bipolar disorder can have “acute” episodes which are characterized by abrupt and large increases in psychotic symptoms. Often, these episodes occur after a period of non-compliance with medication. Both are generally treated with one or more classes of psychotropic medications. Atypical antipsychotic drugs treat psychosis and mood disturbances. Additionally, mood stabilizers such as lithium or valproate treat the manic phase of bipolar disorder, and antidepressants and atypical antipsychotic drugs target the depressive phase. Antipsychotics and mood stabilizers are often used together for “maintenance” treatment to prevent relapse.

A. Schizophrenia (SZ)

SZ is considered a clinical syndrome, and is probably a constellation of several pathologies. Substantial heterogeneity is seen between cases; this is thought to reflect multiple overlapping etiologic factors, including both genetic and environmental contributions. A diagnosis of SZ is typically indicated by chronic psychotic symptoms, e.g., hallucinations and delusions. Disorganization of thought and behavior are common and are considered distinguishing factors in the diagnosis of SZ. Patients typically have some subtle impairment in cognition. Reduced emotional experience and expression, low drive, and impaired speech are observed in a subgroup of patients. Cognitive, emotional and social impairments often appear early in life, while the psychotic symptoms typically manifest in late adolescence or early adulthood in men, a little later in women.

A diagnosis of SZ can be made according to the criteria reported in the Diagnostic and Statistical Manual of Mental Disorders, Fourth Edition, Text Revision, American Psychiatric Association, 2000 (referred to herein as DSM-IV) as follows:

Diagnostic Criteria for SZ—All six criteria must be met for a diagnosis of SZ.

A. Characteristic Symptoms:

Two (or more) of the following, each present for a significant portion of time during a one month period (or less if successfully treated):

(1) delusions; (2) hallucinations; (3) disorganized speech (e.g., frequent derailment or incoherence); (4) grossly disorganized or catatonic behavior; (5) negative symptoms, e.g., affective flattening, alogia, or avolition.

Only one criterion A symptom is required if delusions are bizarre or hallucinations consist of a voice keeping up a running commentary on the person's behavior or thoughts, or two or more voices conversing with each other.

B. Social/Occupational Dysfunction:

For a significant portion of the time since the onset of the disturbance, one or more major areas of functioning such as work, interpersonal relations, or self-care are markedly below the level achieved prior to the onset (or when the onset is in childhood or adolescence, failure to achieve expected level of interpersonal, academic, or occupational achievement).

C. Duration:

Continuous signs of the disturbance persist for at least 6 months. This 6-month period must include at least 1 month of symptoms (or less if successfully treated) that meet Criterion A (i.e., active-phase symptoms) and may include periods of prodromal or residual symptoms. During these prodromal or residual periods, the signs of the disturbance may be manifested by only negative symptoms or two or more symptoms listed in Criterion A present in an attenuated form (e.g., odd beliefs, unusual perceptual experiences).

D. Schizoaffective and Mood Disorder Exclusion:

Schizoaffective Disorder and Mood Disorder With Psychotic Features have been ruled out because either (1) no major depressive, manic, or mixed episodes have occurred concurrently with the active-phase symptoms; or (2) if mood episodes have occurred during active-phase symptoms, their total duration has been brief relative to the duration of the active and residual periods.

E. Substance/General Medical Condition Exclusion:

The disturbance is not due to the direct physiological effects of a substance (e.g., a drug of abuse, a medication) or a general medical condition.

F. Relationship to a Pervasive Developmental Disorder:

If the patient has a history of Autistic Disorder or another Pervasive Developmental Disorder, the additional diagnosis of SZ is made only if prominent delusions or hallucinations are also present for at least a month (or less if successfully treated).

B. Schizoaffective Disorder (SD)

SD is characterized by the presence of affective (depressive or manic) symptoms and schizophrenic symptoms within the same, uninterrupted episode of illness.

The DSM-IV Criteria for a diagnosis of schizoaffective disorder is as follows:

An uninterrupted period of illness during which, at some time, there is either (1) a Major Depressive Episode (which must include depressed mood), (2) a Manic Episode, or (3) a Mixed Episode, concurrent with symptoms that meet (4) Criterion A for SZ, above.

### A. Criteria for Major Depressive Episode

At least five of the following symptoms must be present during the same 2-week period and represent a change from previous functioning; at least one of the symptoms is either (1) depressed mood or (2) loss of interest or pleasure.

(1) depressed mood most of the day, nearly every day, as indicated by either subjective report (e.g., feels sad or empty) or observation made by others (e.g., appears tearful). In children and adolescents, this can be an irritable mood.
(2) markedly diminished interest or pleasure in all, or almost all, activities most of the day, nearly every day (as indicated by either subjective account or observation made by others)
(3) significant weight loss when not dieting or weight gain (e.g., a change of more than 5% of body weight in a month), or decrease or increase in appetite nearly every day. (In children, failure to make expected weight gains is considered).
(4) insomnia or hypersomnia nearly every day
(5) psychomotor agitation or retardation nearly every day (observable by others, not merely subjective feelings of restlessness or being slowed down)
(6) fatigue or loss of energy nearly every day
(7) feelings of worthlessness or excessive or inappropriate guilt (which may be delusional) nearly every day (not merely self-reproach or guilt about being sick)
(8) diminished ability to think or concentrate, or indecisiveness, nearly every day (either by subjective account or as observed by others)
(9) recurrent thoughts of death (not just fear of dying), recurrent suicidal ideation without a specific plan, or a suicide attempt or a specific plan for committing suicide

In addition, the symptoms do not meet criteria for a Mixed Episode. The symptoms cause clinically significant distress or impairment in social, occupational, or other important areas of functioning. The symptoms are not due to the direct physiological effects of a substance (e.g., a drug of abuse, a medication) or a general medical condition (e.g., hypothyroidism).

The symptoms are not better accounted for by Bereavement, i.e., after the loss of a loved one, the symptoms persist for longer than 2 months, or are characterized by marked functional impairment, morbid preoccupation with worthlessness, suicidal ideation, psychotic symptoms, or psychomotor retardation.

### B. Criteria for Manic Episode

A manic episode is a distinct period of abnormally and persistently elevated, expansive, or irritable mood, lasting at least one week (or any duration, if hospitalization is necessary).

During the period of mood disturbance, three (or more) of the following symptoms have persisted (four if the mood is only irritable) and have been present to a significant degree:
(1) inflated self-esteem or grandiosity
(2) decreased need for sleep (e.g., feels rested after only 3 hours of sleep)
(3) more talkative than usual or pressure to keep talking
(4) flight of ideas or subjective experience that thoughts are racing
(5) distractibility (i.e., attention too easily drawn to unimportant or irrelevant external stimuli)
(6) increase in goal-directed activity (either socially, at work or school, or sexually) or psychomotor agitation
(7) excessive involvement in pleasurable activities that have a high potential for painful consequences (e.g., engaging in unrestrained buying sprees, sexual indiscretions, or foolish business investments)

The symptoms do not meet criteria for a Mixed Episode. The mood disturbance is sufficiently severe to cause marked impairment in occupational functioning or in usual social activities or relationships with others, or to necessitate hospitalization to prevent harm to self or others, or there are psychotic features. The symptoms are not due to the direct physiological effects of a substance (e.g., a drug of abuse, a medication, or other treatment) or a general medical condition (e.g., hyperthyroidism).

### C. Criteria for Mixed Episode

A mixed episode occurs when the criteria are met both for a Manic Episode and for a Major Depressive Episode (except for duration) nearly every day during at least a 1-week period. The mood disturbance is sufficiently severe to cause marked impairment in occupational functioning or in usual social activities or relationships with others, or to necessitate hospitalization to prevent harm to self or others, or there are psychotic features.

The symptoms are not due to the direct physiological effects of a substance (e.g., a drug of abuse, a medication, or other treatment) or a general medical condition (e.g., hyperthyroidism).

### D. Criterion A of SZ

See above.

### E. Types of SD

The type of SD may be may be specifiable, as either Bipolar Type, if the disturbance includes a Manic or a Mixed Episode (or a Manic or a Mixed Episode and Major Depressive Episodes), or Depressive Type, if the disturbance only includes Major Depressive Episodes.

### F. Associated Features

Features associated with SD include Learning Problems, Hypoactivity, Psychotic, Euphoric Mood, Depressed Mood, Somatic/Sexual Dysfunction, Hyperactivity, Guilt/Obsession, Odd/Eccentric/Suspicious Personality, Anxious/Fearful/Dependent Personality, and Dramatic/Erratic/Antisocial Personality.

C. Schizotypal Personality Disorder (SPD)

A diagnosis of SPD under the criteria of the DSM-IV is generally based on a pervasive pattern of social and interpersonal deficits marked by acute discomfort with, and reduced capacity for, close relationships as well as by cognitive or perceptual distortions and eccentricities of behavior, beginning by early adulthood and present in a variety of contexts, as indicated by five (or more) of the following:

(1) ideas of reference (excluding delusions of reference)
(2) odd beliefs or magical thinking that influences behavior
(3) inconsistent with subcultural norms (e.g., superstitiousness, belief in clairvoyance, telepathy, or “sixth sense;” in children and adolescents, bizarre fantasies or preoccupations)
(4) unusual perceptual experiences, including bodily illusions
(5) odd thinking and speech (e.g., vague, circumstantial, metaphorical, overelaborate, or stereotyped)
(6) suspiciousness or paranoid ideation
(7) inappropriate or constricted affect
(8) behavior or appearance that is odd, eccentric, or peculiar
(9) lack of close friends or confidants other than first-degree relatives
(10) excessive social anxiety that does not diminish with familiarity and tends to be associated with paranoid fears rather than negative judgments about self

SPD is diagnosed if the symptoms do not occur exclusively during the course of SZ, a Mood Disorder With Psychotic Features, another Psychotic Disorder, or a Pervasive Developmental Disorder, and the disturbance is not due to the direct physiological effects of a substance (e.g., a drug of abuse, a medication) or a general medical condition.

Associated features of SPD include Depressed Mood and Odd/Eccentric/Suspicious Personality.

D. Bipolar Disorder (BD)

Bipolar disorder is also known as manic-depression or manic-depressive disorder. This condition is characterized by mood that alternates between two emotional extremes, or poles: the sadness of depression and the euphoria of mania (see symptoms of mania below).

Between these emotional swings, there are periods when a person's mood is quite normal. When a person is in the depressed phase of bipolar illness, he or she will have the same symptoms as those found in major depressive disorder. The depressive episodes can often be severe. While in a manic phase, a person experiences mood that is extremely elevated, expansive, or irritable. Mania can seriously impair one's normal judgment. When manic, a person is prone towards reckless and inappropriate behavior such as engaging in wild spending sprees or having promiscuous sex. He or she may not be able to realize the harm of his/her behavior and may even lose touch with reality.

There are two primary types of bipolar disorder:

Bipolar I Disorder is diagnosed when a person has had at least one manic or mixed episode, often along with a major depressive episode. It affects equal numbers of men and women in approximately 0.4% to 1.6% of the population.

Bipolar II Disorder is diagnosed when a person has had a major depressive episode along with at least one hypomanic episode. It affects more women than men in about 0.5% of the population.

People with bipolar disorder experience a wide range of feelings depending on the phase of the illness is present. During a phase of depression, a person will have many of the symptoms of a major depressive episode. He or she may have despondent mood, a loss of energy, feelings of worthlessness or guilt, or problems with concentration. Thoughts of suicide are not uncommon. In fact, 10% to 15% of those with bipolar disorder may die by suicide. If the depression is severe, a person may need to be hospitalized for his or her own safety. For those who go through a phase of hypomania, the experience usually feels quite good. If a person's mood and spirit lightens, he or she will be more outgoing and notice more energy and enhanced self-esteem. Lots of ideas come with ease and a person may feel compelled towards greater activity and productivity. A person in a hypomanic phase may also feel more powerful and omnipotent.

The manic phase is the most extreme part of bipolar disorder. A person becomes euphoric, ideas come much too fast, and concentration is nearly impossible. Anger, irritability, fear, and a sense of being out of control are overwhelming A person's judgment is impaired, and he or she may behave recklessly without a sense of consequence. Some people lose touch with reality and experience delusions and hallucinations. When this happens, people often need to be hospitalized for their own safety. If a person with bipolar disorder experiences a severe manic episode, he or she may be abusive to children, spouses, or engage in other violent behaviors. There may also be problems with attendance and performance at school or work, as well as significant difficulties in personal relationships.

The cycles of bipolar disorder may be different for each person. Oftentimes a person may first experience depression. Then depression may be replaced with manic symptoms and the cycle between depression and mania may continue for days, weeks, or months. Between phases of depression and mania some people return to their normal mood. Some others have several periods of either depression or mania. Still others may experience several bouts of depression with infrequent phases of hypomania, or repeated manic episodes with occasional depressive periods. A portion of people, roughly 10% to 20% may only experience mania, while others can have both depression and mania at the same time.

For at least 90% of those who have bipolar disorder the condition is recurrent. They will experience future symptoms of the cycles of mania and depression. Approximately 60%-70% of manic episodes may happen just before or after a depressive episode, and this pattern may happen in a particular way for each person. Most people return to a regular level of functioning between episodes, while some (about 20%-30%) may continue to have some problems with mood stability and social and occupational functioning.

Bipolar I disorder affects equal numbers of males and females, however there does appear to be a gender difference in the onset of the illness. Females are more likely to experience a first episode of depression, while males tend to have a first episode that is manic. Women who have bipolar I or II disorder and who have children may be at a higher risk of experiencing bipolar episodes within several months of giving birth.

A first episode of mania is most likely to occur when a person is in his/her teens or twenties. If a person develops bipolar disorder for the first time after 40 years of age, he or she should be evaluated for the possibility of a medical illness or substance use.

People who have immediate relatives with bipolar I disorder have a higher risk of developing a mood disorder themselves. For these people the rate of developing bipolar II disorder or major depression is 4%-24% and bipolar I disorder is 1%-5%.

Of adolescents who have recurrent major depressive episodes, about 10%-15% of them will likely develop bipolar disorder.

### Diagnostic Criteria of Bipolar I Disorder

Summarized from the Diagnostic and Statistical Manual of Mental Disorders-Fourth Edition

A. A person experiences a current or recent episode that is manic, hypomanic, mixed, or depressed.

To be a manic episode, for at least one week a person's mood must be out of the ordinary and continuously heightened, exaggerated, or irritable.

At least three of the following seven symptoms have been significant and enduring. If the mood is only irritable, then four symptoms are required.

Self-esteem is excessive or grandiose.
The need for sleep is greatly reduced.
Talks much more than usual.
Thoughts and ideas are continuous and without a pattern or focus.
Easily distracted by unimportant things.
An increase in purposeful activity or productivity, or behaving and feeling agitated.
Reckless participation in enjoyable activities that create a high risk for negative consequences (e.g., extensive spending sprees, sexual promiscuity).

The persons' symptoms do not indicate a mixed episode.

The person's symptoms are a cause of great distress or difficulty in functioning at home, work, or other important areas. Or, the symptoms require the person to be hospitalized to protect the person from harming himself/herself or others. Or, the symptoms include psychotic features (hallucinations, delusions).

The person's symptoms are not caused by substance use (e.g., alcohol, drugs, medication), or a medical disorder.

B. Unless this is a first single manic episode there has been at least one manic, mixed, hypomanic, or depressive episode.

For a major depressive episode a person must have experienced at least five of the nine symptoms below for the same two weeks or more, for most of the time almost every day, and this is a change from his/her prior level of functioning. One of the symptoms must be either (a) depressed mood, or (b) loss of interest.

Depressed mood. For children and adolescents, this may be irritable mood.

A significantly reduced level of interest or pleasure in most or all activities.

A considerable loss or gain of weight (e.g., 5% or more change of weight in a month when not dieting). This may also be an increase or decrease in appetite. For children, they may not gain an expected amount of weight.

Difficulty falling or staying asleep (insomnia), or sleeping more than usual (hypersomnia).

Behavior that is agitated or slowed down. Others should be able to observe this.

Feeling fatigued, or diminished energy.

Thoughts of worthlessness or extreme guilt (not about being ill).

Ability to think, concentrate, or make decisions is reduced.

Frequent thoughts of death or suicide (with or without a specific plan), or attempt of suicide.

The persons' symptoms do not indicate a mixed episode.

The person's symptoms are a cause of great distress or difficulty in functioning at home, work, or other important areas.

The person's symptoms are not caused by substance use (e.g., alcohol, drugs, medication), or a medical disorder.

The person's symptoms are not due to normal grief or bereavement over the death of a loved one, they continue for more than two months, or they include great difficulty in functioning, frequent thoughts of worthlessness, thoughts of suicide, symptoms that are psychotic, or behavior that is slowed down (psychomotor retardation).

C. Another disorder does not better explain the episode.

### Diagnostic Criteria of Bipolar II Disorder

Summarized from the Diagnostic and Statistical Manual of Mental Disorders-Fourth Edition

A. The person currently has, or in the past has had at least one major depressive episode:

For a major depressive episode a person must have experienced at least five of the nine symptoms below for the same two weeks or more, for most of the time almost every day, and this is a change from his/her prior level of functioning. One of the symptoms must be either (a) depressed mood, or (b) loss of interest.

Depressed mood. For children and adolescents, this may be irritable mood.

A significantly reduced level of interest or pleasure in most or all activities.

A considerable loss or gain of weight (e.g., 5% or more change of weight in a month when not dieting). This may also be an increase or decrease in appetite. For children, they may not gain an expected amount of weight.

Difficulty falling or staying asleep (insomnia), or sleeping more than usual (hypersomnia).

Behavior that is agitated or slowed down. Others should be able to observe this.

Feeling fatigued, or diminished energy.

Thoughts of worthlessness or extreme guilt (not about being ill).

Ability to think, concentrate, or make decisions is reduced.

Frequent thoughts of death or suicide (with or without a specific plan), or attempt of suicide.

The persons' symptoms do not indicate a mixed episode.

The person's symptoms are a cause of great distress or difficulty in functioning at home, work, or other important areas.

The person's symptoms are not caused by substance use (e.g., alcohol, drugs, medication), or a medical disorder.

The person's symptoms are not due to normal grief or bereavement over the death of a loved one, they continue for more than two months, or they include great difficulty in functioning, frequent thoughts of worthlessness, thoughts of suicide, symptoms that are psychotic, or behavior that is slowed down (psychomotor retardation).

B. The person currently has, or in the past has had at least one hypomanic episode:

For a hypomanic episode a person's mood must be out of the ordinary and continuously heightened, exaggerated, or irritable for at least four days.

At least three of the following seven symptoms have been significant and enduring. If the mood is only irritable, then four symptoms are required.

Self-esteem is excessive or grandiose.

The need for sleep is greatly reduced.

Talks much more than usual.

Thoughts and ideas are continuous and without a pattern or focus.

Easily distracted by unimportant things.

An increase in purposeful activity or productivity, or behaving and feeling agitated.

Reckless participation in enjoyable activities that create a high risk for negative consequences (e.g., extensive spending sprees, sexual promiscuity).

The episode is a substantial change for the person and uncharacteristic of his or her usual functioning.

The changes of functioning and mood can be observed by others.

The person's symptoms are NOT severe enough to cause difficulty in functioning at home, work, or other important areas. Also, the symptoms neither require the person to be hospitalized, nor are there any psychotic features.

The person's symptoms are not caused by substance use (e.g., alcohol, drugs, medication), or a medical disorder. C. The person has never experienced a manic or mixed episode. D. Another disorder does not better explain the episode. E. The symptoms are a cause of great distress or difficulty in functioning at home, work, or other important areas.

E. Psychiatric Endophenotypes in SZ

A number of endophenotypes, i.e., intermediate phenotypes, that may more closely reflect biological mechanisms behind SZ, have been suggested, such as prepulse inhibition, structural abnormalities evident in MRI scans, specific domains of cognition (e.g., executive function), fine motor performance, working memory, etc.

Endophenotypes can also include clinical manifestations such as hallucinations, paranoia, mania, depression, obsessive-compulsive symptoms, etc., as well as response or lack of response to drugs and comorbidity for substance and alcohol abuse. See, e.g., Kendler et al. (1995); Gottesman and Gould (2003); Cadenhead, 2002; Gottesman and Gould (2003); Heinrichs (2004); and Zobel and Maier (2004). There is now evidence that some candidate genes that were identified using DSM-IV type categorical definitions for “affected” individuals may influence specific endophenotypes, see, e.g., Baker et al. (2005); Cannon et al. (2005); Gothelf et al. (2005); Hallmayer et al. (2005); Callicott et al. (2005); Gornick et al. (2005).

F. Use of PANSS (Positive and Negative Syndrome Scale) score for Differential Diagnosis and Evaluation of Clinical Response

The Positive and Negative Syndrome Scale (PANSS) is a comprehensive psychometric scale used to classify psychopathology for severe neuropsychiatric diseases, including SZ. It measures a number of psychiatric endophenotypes or dimensions using quantitative scales based on the scoring of patients by clinicians. It is widely used to classify patients into specific subtypes, and is commonly used for measuring the improvement of symptoms in response to clinical interventions (Kay et al., 1987; Kay et al., 1989; Leucht et al., 2005).

Detailed information on PANSS and Scoring Criteria can be found in the art, e.g., on the world wide web at panss.org, or in the book by Kay (1991) which is incorporated herein in its entirety by reference. Based on these sources, the methodology is summarized briefly below.

PANSS comprises 30 individual subscales. Seven constitute a Positive Symptom Scale, seven the Negative Symptom Scale, and the remaining 16 items make up a General Psychopathology Scale. The scores for these scales are arrived at by summation of ratings across component items. Therefore, the potential ranges are 7 to 49 for the Positive and Negative Scales, and 16 to 112 for the General Psychopathology Scale (Source: The PANSS Institute).

Each of the 30 items is accompanied by a specific definition as well as detailed anchoring criteria for all seven rating points. These seven points represent increasing levels of psychopathology, as follows:

1—absent
2—minimal
3—mild
4—moderate
5—moderate severe
6—severe
7—extreme

The PANSS Individual subscales are described below.

P1. DELUSIONS—Beliefs which are unfounded, unrealistic and idiosyncratic.

P2. CONCEPTUAL DISORGANISATION—Disorganized process of thinking characterized by disruption of goal-directed sequencing, e.g., circumstantiality, loose associations, tangentiality, gross illogicality or thought block.

P3. HALLUCINATORY BEHAVIOUR—Verbal report or behaviour indicating perceptions which are not generated by external stimuli. These may occur in the auditory, visual, olfactory or somatic realms.

P4. EXCITEMENT—Hyperactivity as reflected in accelerated motor behaviour, heightened responsivity to stimuli, hypervigilance or excessive mood lability.

P5. GRANDIOSITY—Exaggerated self-opinion and unrealistic convictions of superiority, including delusions of extraordinary abilities, wealth, knowledge, fame, power and moral righteousness.

P6. SUSPICIOUSNESS/PERSECUTION—Unrealistic or exaggerated ideas of persecution, as reflected in guardedness, ad distrustful attitude, suspicious hypervigilance or frank delusions that others mean harm.

P7. HOSTILITY—Verbal and nonverbal expressions of anger and resentment, including sarcasm, passive-aggressive behavior, verbal abuse and assualtiveness.

N1. BLUNTED AFFECT—Diminished emotional responsiveness as characterized by a reduction in facial expression, modulation of feelings and communicative gestures.

N2. EMOTIONAL WITHDRAWAL—Lack of interest in, involvement with, and affective commitment to life's events.

N3. POOR RAPPORT—Lack of interpersonal empathy, openness in conversation and sense of closeness, interest or involvement with the interviewer. This is evidenced by interpersonal distancing and reduced verbal and nonverbal communication.

N4. PASSIVE/APATHETIC SOCIAL WITHDRAWAL—Diminished interest and initiative in social interactions due to passivity, apathy, anergy or avolition. This leads to reduced interpersonal involvements and neglect of activities of daily living.

N5. DIFFICULTY IN ABSTRACT THINKING—Impairment in the use of the abstract-symbolic mode of thinking, as evidenced by difficulty in classification, forming generalizations and proceeding beyond concrete or egocentric thinking in problem-solving tasks.

N6. LACK OF SPONTANEITY AND FLOW OF CONVERSATION—Reduction in the normal flow of communication associated with apathy, avolition, defensiveness or cognitive deficit. This is manifested by diminished fluidity and productivity of the verbal interactional process.

N7. STEREOTYPED THINKING—Decreased fluidity, spontaneity and flexibility of thinking, as evidenced in rigid, repetitious or barren thought content.

G1. SOMATIC CONCERN—Physical complaints or beliefs about bodily illness or malfunctions. This may range from a vague sense of ill being to clear-cut delusions of catastrophic physical disease.

G2. ANXIETY—Subjective experience of nervousness, worry, apprehension or restlessness, ranging from excessive concern about the present or future to feelings of panic.

G3. GUILT FEELINGS—Sense of remorse or self-blame for real or imagined misdeeds in the past.

G4. TENSION—Overt physical manifestations of fear, anxiety, and agitation, such as stiffness, tremor, profuse sweating and restlessness.

G5. MANNERISMS AND POSTURING—Unnatural movements or posture as characterized be an awkward, stilted, disorganized, or bizarre appearance.

G6. DEPRESSION—Feelings of sadness, discouragement, helplessness and pessimism.

G7. MOTOR RETARDATION—Reduction in motor activity as reflected in slowing or lessening or movements and speech, diminished responsiveness of stimuli, and reduced body tone.

G8. UNCOOPERATIVENESS—Active refusal to comply with the will of significant others, including the interviewer, hospital staff or family, which may be associated with distrust, defensiveness, stubbornness, negativism, rejection of authority, hostility or belligerence.

G9. UNUSUAL THOUGHT CONTENT—Thinking characterized by strange, fantastic or bizarre ideas, ranging from those which are remote or atypical to those which are distorted, illogical and patently absurd.

G10. DISORIENTATION—Lack of awareness of one's relationship to the milieu, including persons, place and time, which may be due to confusion or withdrawal.

G11. POOR ATTENTION—Failure in focused alertness manifested by poor concentration, distractibility from internal and external stimuli, and difficulty in harnessing, sustaining or shifting focus to new stimuli.

G12. LACK OF JUDGEMENT AND INSIGHT—Impaired awareness or understanding of one's own psychiatric condition and life situation. This is evidenced by failure to recognize past or present psychiatric illness or symptoms, denial of need for psychiatric hospitalization or treatment, decisions characterized by poor anticipation or consequences, and unrealistic short-term and long-range planning.

G13. DISTURBANCE OF VOLITION—Disturbance in the willful initiation, sustenance and control of one's thoughts, behavior, movements and speech.

G14. POOR IMPULSE CONTROL—Disordered regulation and control of action on inner urges, resulting in sudden, unmodulated, arbitrary or misdirected discharge of tension and emotions without concern about consequences.

G15. PREOCCUPATION—Absorption with internally generated thoughts and feelings and with autistic experiences to the detriment of reality orientation and adaptive behavior.

G16. ACTIVE SOCIAL AVOIDANCE—Diminished social involvement associated with unwarranted fear, hostility, or distrust.

Each patient's disease manifestation and process is unique. PANSS provides a structured, objective way of describing the various aspects of psychopathology of a given patient. However, proper implementation of the PANSS requires highly trained personnel to conduct the assessment and to interpret the results, and there is potential for site to site variability, especially outside the research setting.

Each of the PANSS composite scales and subscales can be considered a clinical endophenotype. The ability to link genetic profiles to these clinical endophenotypes changes as response to psychotic treatments, or severity of the diseases, will enable clinicians to refine a patient's diagnosis and develop a personalized therapeutic strategy for each patient. By identifying the genetic contributions to specific endophenotypes, the physician can create a personalized diagnosis and treatment regime for the patient.

Additionally, changes in PANSS or the Brief Psychiatric Rating Scale (BPRS), which is derived from PANSS, are often the primary measures of efficacy in clinical trials. Moreover, certain subscales and composite scores are also evaluated for change. For example, positive symptoms and negative symptoms are two composite scores that have clinical relevance.

### IV. TREATMENT OF PSYCHOTIC DISORDERS

A. Current Treatments for Psychotic Disorders

Atypical antipsychotic drugs listed in order of current prescribing frequency for these disorders include: risperidone, quetiapine, olanzapine, aripiprazole, ziprasidone, clozapine, paliperidone, and iloperidone. Typical antipsychotic drugs include haloperidol, fluphenazine, perphenazine, and others (Meltzer and Bobo, 2009). Atypical antipsychotics are favored because of lower parkinsonian side effects and perceived greater efficacy. There are several long acting formulations of typical antipsychotic drugs, which are seldom used in the US. Among the atypical agents, only long acting, injectable risperidone, paliperidone and olanzapine are currently available; and others, e.g., long acting aripiprazole, are in development. These formulations are highly effective since they provide great help with regard to compliance, an enormous problem with both bipolar and schizophrenia patients. However, they are underutilized in the marketplace.

All of the antipsychotic medications have some degree of side-effects and other limitations. Metabolic side effects with some atypical antipsychotic drugs (weight gain, increased lipids, and problems with glucose regulation, including diabetes) exacerbate the discontinuation rates and limit overall effectiveness the implicated drugs. Olanzapine and clozapine, two of the most efficacious drugs for treating psychosis, have the greatest incidence of metabolic side-effects (Nasrallah, 2007).

Subjects with SZ typically require acute treatment for psychotic exacerbations, and long-term treatment including maintenance and prophylactic strategies to sustain symptom improvement and prevent recurrence of psychosis. Subjects with schizoaffective disorder experience the symptoms of both SZ and affective disorder (manic and/or depressive), thus require the specific treatments for each disorder. Subjects with SPD sometimes require medication for acute psychotic episodes but are often treated using psychosocial methods. The methods described herein can include the administration of one or more accepted or experimental treatment modalities to a person identified as having, suspected to have, or at risk of developing a psychotic disorder such as SZ, SPD, or a SD, based on the presence of a GARP genetic signature status. Currently accepted treatments presently include both pharmacologic and psychosocial management, and occasionally electroconvulsive therapy (ECT).

Standard pharmacologic therapies for SZ and SD include the administration of one or more antipsychotic medications, which are typically antagonists acting at postsynaptic D₂ dopamine receptors in the brain. Antipsychotic medications include conventional, or first generation, antipsychotic agents, which are sometimes referred to as neuroleptics because of their neurologic side effects, and second generation antipsychotic agents, which are less likely to exhibit neuroleptic effects and have been termed atypical antipsychotics.

Standard pharmacologic therapies for SD also include the administration of a combination of antidepressant, and anti-anxiety medication. Suitable antidepressants include serotonergic antidepressants, e.g., fluoxetine or trazodone. Suitable anxiolytics include benzodiazepines, e.g., lorazepam, clonazepam. Lithium can also be administered. Thus, in some embodiments, the methods can include the administration of one or more antidepressant and/or anti-anxiety medications to a person identified as having or suspected to have SD in combination with a treatment plan based on GARP genetic signature status.

The methods can also include psychosocial and rehabilitation interventions, e.g., interventions that are generally accepted as therapeutically beneficial, e.g., cognitive-behavioral therapy for treatment-resistant positive psychotic symptoms; supportive, problem-solving, educationally oriented psychotherapy; family therapy and education programs aimed at helping patients and their families understand the patient's illness, reduce stress, and enhance coping capabilities; social and living skills training; supported employment programs; and/or the provision of supervised residential living arrangements.

Currently accepted treatments for SZ are described in greater detail in the Practice Guideline for the Treatment of Patients With Schizophrenia, 2004, which is incorporated herein by reference in its entirety.

Currently accepted treatments for BD are described in greater detail in the Treatment of Patients With Bipolar Disorder, 2006, which is incorporated herein by reference in its entirety.

There are seven commonly prescribed atypical antipsychotic medications for bipolar disorder:

Abilify (aripiprazole)
Risperdal (risperidone)
Zyprexa (olanzapine)
Seroquel (quetiapine)
Geodon (ziprasidone)
Clozaril (clozapine)
Symbyax (olanzapine/fluoxetine)

The atypical antipsychotics have several significant side-effects, including weight gain, metabolic and hormonal disregulation, and sexual dysfunction. Weight gain, in particular, can be a significant issue as many people treated with atypical antipsychotics expect to gain weight, sometimes significantly so. Because weight gain is also associated with an increased risk for Type II diabetes, individuals taking an atyptical antipsychotic should be carefully monitored by their physician. Other metabolic disturbances include elevated prolactin levels and altered steroid metabolism. Additionally, both men and women report significant levels of sexual dysfunction.

Drugs approved for the treatment of Bipolar disorder can have one or more indications, e.g., U.S. FDA (or other regulatory body) approved uses. Some of these indications include: Treatment of Acute Mania episodes; Bipolar Mania maintenance; Bipolar depression; Treatment of mixed mania and depression episodes.

Clinicians most commonly initiate treatment with oral risperidone, especially now that it is generic. Often, however, clinicians will switch patients to another drug after less than 1 to 6 months treatment. This common practice occurs based on what is perceived by patients and clinicians as both insufficient efficacy and unacceptable side effects, such as Parkinsonism, prolactin elevations or weight gain. Olanzapine, which was found to be the most effective treatment in the CATIE study and is closest in pharmacology to clozapine (the drug generally accepted to have the highest efficacy, particularly in treatment resistant subjects), is considered to be a highly effective agent (Meltzer and Bobo, 2009). Indeed, olanzapine became the most prescribed treatment and initial drug of choice after its introduction until concerns about metabolic side effects became evident (Meltzer, 2005). Metabolic side effects (weight gain, glucose dysregulation, lipid dysregulation and risk of diabetes) have greatly reduced the utilization of olanzapine despite its high efficacy.

B. Pharmacogenetic Testing

Clinicians currently have no way of knowing which drug is best for a specific patient other than from previous successful or unsuccessful trials. They make decisions based on their overall experience with a given drug as well as the influence of advertising and recommendations from clinic supervisors. Also options may be limited because, without a medical rationale, many states limit access to highly efficacious medications (primarily olanzapine) for Medicaid recipients due to cost concerns.

Payers would welcome a diagnostic test that enhances compliance and response rates for their clients. Compliance with oral antipsychotic drugs is a major problem, with 50% of patients ceasing to take them within 6 months of prescription, leading to relapse (return of psychosis) and hospitalization. While U.S. consumers and government agencies spend a considerable amount of money on atypical drugs, relapse and hospitalization represent the largest costs associated with schizophrenia and BP. Thus, reducing the rate of hospitalizations and relapse can lead to very significant cost savings and improved patient care.

It is consensus opinion among experts in the treatment of schizophrenia that a pharmacogenetic test which could deliver information that led to a more rapid and extensive control of psychosis would become the dominant driver of choice of medication options. Physicians could engage patients in a rational dialogue regarding treatment choice based on genetic profile. With this additional information, physicians and patients could make calculated risk-benefit analysis regarding potential efficacy and side-effect concerns of the various antipsychotic medications. There is also a published cost-effectiveness analysis which supports the value of pharmacogenetic testing (Perlis et al., 2005).

With regards to both prophylactic and therapeutic methods of treatment of psychotic disorders such as a psychotic disorder, such treatments may be specifically tailored or modified, based on knowledge obtained from the field of pharmacogenomics. “Pharmacogenomics,” as used herein, refers to the application of genomics technologies such as structural chromosomal analysis, to drugs in clinical development and on the market. See, for example, Eichelbaum et al. (1996) and Linder et al. (1997). Specifically, as used herein, the term refers the study of how a patient's genes determine his or her response to a drug (e.g., a patient's “drug response phenotype,” or “drug response genotype”). Thus, another aspect of the invention provides methods for tailoring an individual's prophylactic or therapeutic treatment according to that individual's drug response genotype, especially, a GARP genetic signature status.

Information generated from pharmacogenomic research using a method described herein can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when administering a therapeutic composition, e.g., a cytotoxic agent or combination of cytotoxic agents, to a patient, as a means of treating or preventing psychotic disorders such as a psychotic disorder.

In one embodiment, a physician or clinician may consider applying knowledge obtained in relevant pharmacogenomics studies, e.g., using a method described herein, when determining whether to administer a pharmaceutical composition, e.g., an antipsychotic agent or a combination of antipsychotic agents, to a subject. In another embodiment, a physician or clinician may consider applying such knowledge when determining the dosage, e.g., amount per treatment or frequency of treatments, of a treatment, e.g., a antipsychotic agent or combination of antipsychotic agents, administered to a patient.

As one example, information regarding a genetic signature associated with an altered pharmacogenomic response for psychotic disorders as described herein, can be used to stratify or select a subject population for a clinical trial. The information can, in some embodiments, be used to stratify individuals that may exhibit a toxic response to a treatment from those that will not. In other cases, the information can be used to separate those that are more likely to be non-responders from those who will be responders. The GARP genetic signature described herein can be used in pharmacogenomics-based design and to manage the conduct of a clinical trial, e.g., as described in U.S. Pat. Pub. No. 2003/0108938.

As another example, information regarding a GARP genetic signature associated with an increased severity of psychotic disorders, or with altered pharmacogenomic response for psychotic disorders, as described herein, can be used to stratify or select human cells or cell lines for drug testing purposes. Human cells are useful for studying the effect of a polymorphism on physiological function, and for identifying and/or evaluating potential therapeutic agents for the treatment of psychotic disorders, e.g., anti-psychotics. Thus the methods can include performing the present methods on genetic material from a cell line. The information can, in some embodiments, be used to separate cells that respond particular drugs from those that do not respond, e.g. which cells show altered second messenger signaling.

C. Theranostics

As used herein, the word theranostic is a combination of a specific therapy and diagnostic. The combination represents the use of a diagnostic test to identify a specific patient subtype(s) of psychotic disorders such as a psychotic disorder that have common genetic, clinical, metabolic, and/or prognostic features or a patient subtype that has differential drug response. By performing a diagnostic test, e.g. a genetic test to determine GARP genetic signature, the physician or clinician can place the patient into a specific disease sub-type or category, for example, a GARP genetic signature positive or negative subgroup. Moreover, patients in this sub-type respond to a given therapy in a particular manner.

Also included herein are compositions and methods for the identification and treatment of subjects who have an increased severity of a psychotic disorder, or altered clinical presentation of a psychotic disorder, such that a theranostic approach can be taken to test such individuals to determine the effectiveness of a particular therapeutic intervention (e.g., a pharmaceutical or non-pharmaceutical intervention as described herein) and/or to alter the intervention to enhance the effectiveness. Thus, the methods and compositions described herein provide a means of optimizing the treatment of a subject having or suspected to have a psychotic disorder such as SZ. Provided herein is a theranostic approach to treating and preventing a psychotic disorder such as SZ, by integrating diagnostics and therapeutics to improve the real-time treatment of a subject. Practically, this means creating tests that can identify which patients are most suited to a particular therapy, and providing feedback on how well a drug is working to optimize treatment regimens.

Within the clinical trial setting, a theranostic method or composition of the invention can provide key information to optimize trial design, monitor efficacy, and enhance drug safety. For instance, “trial design” theranostics can be used for patient stratification, determination of patient eligibility (inclusion/exclusion), creation of homogeneous treatment groups, and selection of patient samples that are representative of the general population. Such theranostic tests can therefore provide the means for patient efficacy enrichment, thereby minimizing the number of individuals needed for trial recruitment. “Efficacy” theranostics are useful for monitoring therapy and assessing efficacy criteria. Finally, “safety” theranostics can be used to prevent adverse drug reactions or avoid medication error.

The methods described herein can include retrospective analysis of clinical trial data as well, both at the subject level and for the entire trial, to detect correlations between a genetic signature as described herein and any measurable or quantifiable parameter relating to the outcome of the treatment, e.g., efficacy (the results of which may be binary (i.e., yes and no) as well as along a continuum), side-effect profile (e.g., weight gain, metabolic dysfunction, lipid dysfunction, movement disorders, or extrapyramidal symptoms), treatment maintenance and discontinuation rates, return to work status, hospitalizations, suicidality, total healthcare cost, social functioning scales, response to non-pharmacological treatments, and/or dose response curves. The results of these correlations can then be used to influence decision-making, e.g., regarding treatment or therapeutic strategies, provision of services, and/or payment. For example, a correlation between a positive outcome parameter (e.g., high efficacy, low side effect profile, high treatment maintenance/low discontinuation rates, good return to work status, low hospitalizations, low suicidality, low total healthcare cost, high social function scale, favorable response to non-pharmacological treatments, and/or acceptable dose response curves) and a GARP genetic signature can influence treatment such that the treatment is recommended or selected based on the presence or absence of the GARP genetic signature.

### V. KITS

Certain aspects of the present invention provides kits, such as diagnostic and therapeutic kits. Kits may comprise a container with a label. Suitable containers include, for example, bottles, vials, and test tubes. The containers may be formed from a variety of materials such as glass or plastic. The container may hold a composition which includes a probe or an array that could be used to determine a GARP genetic signature, which could be effective for diagnostic or pharmacogenomic applications. The label on the container may indicate that the composition is used for a specific diagnostic or pharmacogenomic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

Within the scope of the invention are kits comprising a probe that hybridizes with a region of human chromosome as described herein and can be used to detect a polymorphism related to a GARP genetic signature. The kit can include one or more other elements including: instructions for use; and other reagents, e.g., a label, or an agent useful for attaching a label to the probe. Instructions for use can include instructions for diagnostic applications of the probe for assessing severity of a psychotic disorder such as SZ in a method described herein. Other instructions can include instructions for attaching a label to the probe, instructions for performing in situ analysis with the probe, and/or instructions for obtaining a sample to be analyzed from a subject. As discussed above, the kit can include a label, e.g., any of the labels described herein. In some embodiments, the kit includes a labeled probe that hybridizes to a region of human chromosome as described herein, e.g., a labeled probe as described herein.

The kit can also include one or more additional probes that hybridize to the same chromosome, e.g., chromosome 2 or 5, or another chromosome or portion thereof that can have an abnormality associated with diagnostic applications. For example, the additional probe or probes can be: a probe that hybridizes to human chromosome 22q11-12 or a portion thereof, (e.g., a probe that detects a sequence associated with a psychotic disorder in this region of chromosome 22), or probes that hybridize to all or a portion of 22q12.3 (e.g., near D22S283), 22q11.2, 22q11.2, 22q11-q13, 1q42.1, 1q42.1, 1q21-q22, 2p, 2q, 3p25, 4p, 4q, 5q11.2-q13.3, 6p22.3, 6p23, 6q13-q26, 7q, 8p12-21, 8q, 9p, 10p15-p13 (e.g., near D10S189), 10q22.3, 11q14-q21, 12q24, 13q34, 13q32, 14q32.3, 15₈15, 16p, 17q, 18p, 18q, 19p. 20p, 21q, Xq, and/or the X/Y pseudoautosomal region. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

Kits for use in self-testing can also be provided. For example, such test kits can include devices and instructions that a subject can use to obtain a sample, e.g., of buccal cells or blood, without the aid of a health care provider. For example, buccal cells can be obtained using a buccal swab or brush, or using mouthwash.

Kits as provided herein can also include a mailer, e.g., a postage paid envelope or mailing pack, that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms, e.g., the test requisition form, and the container holding the sample, can be coded, e.g., with a bar code, for identifying the subject who provided the sample.

Kits may also comprise or be coupled to a system which can make recommendations and/or analysis of efficacy, risk or side effects for treatment of a psychotic disorder based on a determined GARP genetic signature status. The system may comprise a server, a processor, or a tangible computer readable program product. For example, if the kit determines the presence or absence of an GARP genetic signature, the system may perform an analysis and generate an efficacy and risk profile for treatment with a psychotic treatment, such as treating with olanzapine, clozapine, or quetiapine.

### VI. PROBES

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence, or any range derivable therein. The probe sequence must also be sufficiently long so that the probe exhibits selectivity for the target sequence over non-target sequences. For example, the probe will be at least 10, e.g., 15, 20, 25, 30, 35, 50, 100, 200, 300, 400, 500, 600, 700, 800, 900, or more nucleotides in length, or any range derivable therein. In some embodiments, the probes are not more than 30, 50, 100, 200, 300, 500, 750, or 1000 nucleotides in length, or any range derivable therein. Specifically, probes may be about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

In some embodiments, the probe is a test probe, e.g., a probe that can be used to detect polymorphisms in a region described herein, e.g., SV2C or MGAT4C polymorphisms as described herein. In some embodiments, the probe can hybridize to a target sequence in complete linkage disequilibrium with one of the SNPs described herein for determination of an GARP genetic signature.

In some embodiments, the probe can bind to another marker sequence associated with a psychotic disorder such as SZ as described herein.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially, e.g., from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson (1998); Wheeless et al. (1994); U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end), such as rs2285162, rs2285166, or rs2285167, will generally hybridize preferentially to a nucleic acid comprising that sequence, as opposed to a nucleic acid comprising an alternate polymorphic variant.

### VII. ARRAYS AND USES THEREOF

In another aspect, the invention features methods of determining the absence or presence of an GARP genetic signature using an array described above. In a further aspect, the invention features arrays that include a substrate having a plurality of addressable areas, and methods of using them. At least one area of the plurality includes a nucleic acid probe that binds specifically to a sequence comprising a polymorphism such rs11960832 or rs7975477 and can be used to detect the absence or presence of said polymorphism, e.g., one or more SNPs, microsatellites, minisatellites, or indels, to determine a GARP genetic signature. For example, the array can include one or more nucleic acid probes that can be used to detect a polymorphism such as rs11960832 or rs7975477. In some embodiments, the array further includes at least one area that includes a nucleic acid probe that can be used to specifically detect another marker associated with a psychotic disorder such as SZ as described herein. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., Published PCT Application Nos. WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments.

Additional description of use of oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments of the invention.

The methods described herein can include providing an array as described herein; contacting the array with a sample, e.g., a portion of genomic DNA that includes at least a portion of human chromosome 22, e.g., a region between and/or including SNPs for a SULT4A gene, and/or optionally, a different portion of genomic DNA, e.g., a portion that includes a different portion of human chromosomes 1, 2, 3, 4, 5, 6, 7, 8, 9, 10, 11, 12, 13, 14, 15, 16, 17, 18, 19, 20, 21 and/or 22, or another chromosome, e.g., including another region associated with a psychotic disorder, pharmacological response, and/or psychiatric endophenotypes, and detecting binding of a nucleic acid from the sample to the array. Optionally, the method includes amplifying nucleic acid from the sample, e.g., genomic DNA that includes a portion of a human chromosome described herein, and, optionally, a region that includes another region associated with a psychotic disorder, pharmacological response, and/or psychiatric endophenotypes, prior to or during contact with the array.

In some aspects, the methods described herein can include using an array that can ascertain differential expression patterns or copy numbers of one or more genes in samples from normal and affected individuals (see, e.g., Redon et al., 2006). For example, arrays of probes to a marker described herein can be used to measure polymorphisms between DNA from a subject having a psychotic disorder and control DNA, e.g., DNA obtained from an individual that does not have a psychotic disorder and has no familial risk factors for a psychotic disorder. Since the clones on the array contain sequence tags, their positions on the array are accurately known relative to the genomic sequence. Different hybridization patterns between DNA from an individual afflicted with a psychotic disorder such as SZ and DNA from a normal individual at areas in the array corresponding to markers in human chromosome 1, 2, 3, 4, 5, 6, 7, 8, 9, 10, 11, 12, 13, 14, 15, 16, 17, 18, 19, 20, 21 and/or 22 as described herein, and, optionally, one or more other regions associated with a psychotic disorder are indicative of a risk of SZ-spectrum disorders. Methods for array production, hybridization, and analysis are described, e.g., in Snijders et al. (2001); Klein et al. (1999); Albertson et al. (2003); and Snijders et al. (2002). Real time quantitative PCR can also be used to determine copy number.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Assigning GLP1R Antipsychotic Response Phenotype (GARP)

As defined here, GARP is a genetic signature that uses four common haplotypes from the GLP1R gene to determine likely response to various antipsychotic medications. SNP genotypes provided by others were used to establish GLP1R Antipsychotic Response Phenotype (GARP) genetic signature status using a simple and intuitive scoring method based on the genotypes of SNPs rs6923761 (A/G), rs2300615 (T/G), and rs1042044 (A/C). Both rs6923761 and rs1042044 SNPs create protein coding changes, serine (A) vs. glycine (G) and leucine (A) vs. phenylalanine (C) respectively. These SNPs describe four common haplotypes, and four difference response phenotypes as detailed below.

### A. Genotyping Methodology

The genotyping methodologies used for the Examples are well established and of high quality. Both the Affymetrix and Illumina platforms are widely used commercially available platforms. The technology behind both platforms has been included in CLIA-approved genotyping tests at the manufactures' own facilities and at third party providers.

The data from the inventors derived from a large scale genotyping project that used microarray-based whole genome SNP genotyping approaches. The project was performed by the CATIE study group using Affymetrix and Perlegen microarray platforms (Sullivan et al., 2008).

The inventors were provided finished genotype data and do not have access to raw data files such as .cel files. As described in the following examples, the inventors developed a method that uses SNP genotypes to determine and assign GARP genetic signature to individual subjects.

### B. Genotype Determination in the CATIE Study

Details of the genotyping performed by the CATIE consortium, including their rigorous quality control procedures, are described elsewhere (Sullivan et al., 2008). Briefly, peripheral venous blood was collected, and genomic DNA was extracted from lymphocytes. Analyses were performed at Perlegen Sciences in its CLIA, GLP facility using two microarray genotyping systems. The first was the Affymetrix 500K “A” chipset (Nsp I and Sty I chips, Santa Clara, Calif.) used as specified by the manufacturer. Detailed description of the SNP detection methodology can be found in Affymetrix product documentation (available on the world wide web at affymetrix.com/products_services/arrays/specific/500k.affx). Secondly, Perlegen used a custom 164K chip. The genotype calling methodology and extensive quality control measures used by the CATIE study group are reported by Sullivan and coworkers (Sullivan et al., 2008) and further described in detail in the supplemental technical material provided by the CATIE consortium (Sullivan et al., 2009).

As an example of the level of quality control used, the CATIE group performed duplicate analysis on 36 samples. The proportion of SNPs with non-missing genotype calls that disagreed in these duplicated samples was 0.00291. As an additional control, 277 individuals were genotyped at a second facility using a different SNP-calling algorithm to investigate potential site bias. Only 0.73% of called genotypes differed between the two sites.

### C. Assigning GLIP1R Haplotype and Diplotype Status

The three SNPs produce four separate haplotypes that have a frequency of greater than 1% In Caucasians and African Americans. Each person has two copies of the GLP1R gene. Listed in order of appearance on the chromosome, SNPs rs6923761 (A/G), rs2300615 (G/T), and rs1042044 (A/C) define the following four haplotypes as displayed in Table 1. Note that in three of the four cases, a single SNP is sufficient to tag the haplotype (bold font Table A).

TABLE 1A
definition of GLP1R haplotypes

| rs6923761 | rs2300615 | rs1042044 | haplotype |
|---|---|---|---|
| G | T | A | GLP1R-1 |
| A | T | C | GLP1R-2 |
| G | G | C | GLP1R-3 |
| G | T | C | GLP1R-4 |

Since GLP1R has significant linkage disequilibrium between SNPs, the same haplotype information can be captured in multiple ways. For example, the same four haplotypes can be defined using 4 SNPs as shown in Table 1B or using a substantially different set of 6 SNPs as shown in Table 1C.

TABLE 1B
alternative methods of determinig of GLP1R haplotypes

| rs9296283 | rs7766275 | rs2300615 | rs1042044 | haplotype |
|---|---|---|---|---|
| G | A | T | A | GLP1R-1 |
| A | C | T | C | GLP1R-2 |
| A | C | G | C | GLP1R-3 |
| G | A | T | C | GLP1R-4 |

TABLE 1C
alternative methods of determining of GLP1R haplotypes

| rs10305439 | rs742764 | rs2268650 | rs910170 | rs6923761 | rs9296283 | rs7766275 | rs2300615 | rs2235868 | rs1042044 | haplotype |
|---|---|---|---|---|---|---|---|---|---|---|
| C | C | G | A | G | G | A | T | A | A | GLP1R-1 |
| A | T | A | G | A | A | C | T | C | C | GLP1R-2 |
| A | T | A | G | G | A | C | G | C | C | GLP1R-3 |
| A | C | G | G | G | G | A | T | C | C | GLP1R-4 |

These four haplotypes form ten diplotypes (combinations of two haplotypes). Table 2 below enumerates all ten possible, two-haplotype combinations.

TABLE 2
GLP1R diplotypes

| rs6923761 | rs2300615 | rs1042044 | HAPLOTYPE A | HAPLOTYPE B |
|---|---|---|---|---|
| G_G | T_T | A_A | GLP1R-1 | GLP1R-1 |
| A_G | T_T | A_C | GLP1R-1 | GLP1R-2 |
| G_G | G_T | A_C | GLP1R-1 | GLP1R-3 |
| G_G | T_T | A_C | GLP1R-1 | GLP1R-4 |
| A_A | T_T | C_C | GLP1R-2 | GLP1R-2 |
| A_G | G_T | C_C | GLP1R-2 | GLP1R-3 |
| A_G | T_T | C_C | GLP1R-2 | GLP1R-4 |
| G_G | G_G | C_C | GLP1R-3 | GLP1R-3 |
| G_G | G_T | C_C | GLP1R-3 | GLP1R-4 |
| G_G | T_T | C_C | GLP1R-4 | GLP1R-4 |

### D. Assigning Garp Genetic Signatures Based on GLIP1R Haplotype Combinations

While ten possible haplotype combinations exist, the inventors have identified four major response phenotypes, GARP1 through GARP4. A given individual will belong to a specific response type that displays specific pharmacogenetic response to antipsychotic treatments.

TABLE 3
Definition of GARP genetic signatures

| GARP Genetic Signature | HAPLOTYPE A | HAPLOTYPE B |
|---|---|---|
| GARP-1 | GLP1R-1 | GLP1R-1 |
| GARP-2 | GLP1R-2 | GLP1R-1 OR GLP1R-2 OR |
| | | GLP1R-3 OR GLP1R-4 |
| GARP-3 | GLP1R-3 | GLP1R-1 OR GLP1R-3 OR |
| | | GLP1R-4 |
| GARP-4 | GLP1R-4 | GLP1R-1 OR GLP1R-4 |

### Example 2

### The GARP Genetic Signatures Predict Antipsychotic Response

The CATIE study, a large federally funded clinical trial designed to assess the efficacy of antipsychotics in a real world setting, is a valuable resource for determining the role of genes in baseline psychopathology and drug response (Lieberman et al., 2005; Stroup et al., 2003). As part of the CATIE trial, detailed clinical evaluations were conducted, including Positive and Negative Syndrome Scale (PANSS) measurements at multiple time points, cognitive evaluation, vital signs, blood chemistry results, and drug response data. Additionally, whole genome SNP genotyping was performed for roughly half of the trial participants (Sullivan et al., 2008).

### A. Data and Methods Relating to the CATIE Sample

Genotype and phenotype data for the CATIE trial were recently made available to qualified researchers through the NIMH Center for Collaborative Genetic Studies on Mental Disorders. The sample consisted of a total of 738 patients. Further details on the patient population are described in the study by Sullivan and coworkers (Sullivan et al., 2008). The CATIE SNP genotype data evaluated included a total of 492,900 SNPs located throughout the genome.

The design of the CATIE study has been described in detail by others (Lieberman et al., 2005; Stroup et al., 2003). Briefly, 1460 subjects were randomly assigned one of several antipsychotics and those who did not respond or who chose to quit their current medication were re-randomized to another drug. Subjects that failed Phase 1 treatment with the typical antipsychotic perphenazine were treated differently than those subjects that failed phase I treatment with an atypical antipsychotic. Instead of entering into Phase II of the trial, subjects who switched from perphenazine entered Phase IB. For Phase IB, subjects were randomly assigned to an atypical antipsychotic—olanzapine, quetiapine or risperidone. Subjects who failed Phase IB then entered Phase II.

A total of 738 subjects consented to provide DNA for genetic study. Details regarding SNP genotyping and quality control have been recently published (Sullivan et al., 2008). Only retrospective genetic analyses, judged to be exempt from human studies requirements by an IRB, were conducted in the current study. For the genetic analyses presented herein, the inventors combined and evaluated drug response data for Phase IA, Phase IB, and Phase II of the CATIE study. All tested patients were studied using well established clinical endpoints comparing baseline, intermediate, and last observation carried forward (LOCF) for the PANSS scores using CATIE data. LOCF analysis is a commonly used clinical endpoint for clinical trials for neuropsychiatric drugs, with particular changes in PANSS scores being used to indicate clinically significant response (Leucht et al., 2009).

Using the clinical data from the CATIE trial, the inventors derived several different metrics used for quantifying response in clinical trials of antipsychotic medications. The most commonly used psychometric instrument is the Positive and Negative Syndrome Scale (PANSS), a 30-item semi-quantitative instrument scored by clinical professionals (Kay et al., 1987). The greater the decrease in PANSS score, the better the clinical response. The most direct measure of clinical response is absolute change in PANSS score (delta PANSS). In addition to absolute change, the improvement is often reported as a percentage change from baseline PANSS score.

For example, many drug trials for antipsychotic medications report response as quantitative changes in PANSS score (Leucht et al., 2009). This approach is particularly useful for trials with active comparators or in cases where claims of drug superiority or non-inferiority are important (i.e. where relative improvement in symptoms is a key factor).

### B. the GARP Genetic Signatures Predict Response to Antipsychotics Based on Quantitative Change in PANSS Score

As mentioned previously, the mean change in PANSS at LOCF provides an important measure of efficacy in patients undergoing antipsychotic treatments. Indeed, many pivotal trials in the psychosis field used absolute and/or percentage change in PANSS Score as the primary endpoint (Leucht et al., 2009). The performance of the investigational drug is then compared with placebo and/or an active comparator. Therefore, the inventors determined the impact of segmentation by GARP genetic signature status on the quantitative measure of clinical improvement—delta PANSS (for LOCF).

Tables 4, 5, 6, and 7 below show the mean delta PANSS response for each of the drugs used in Phases 1-2 of the CATIE trial. Each table lists the six drugs used during the first two phases of the CATIE trial. Patients are segmented into those that had a specific GARP response type (“positive”) for GARP-1, GARP-2, GARP-3, or GARP-4, in tables 4, 5, 6, and 7, respectively, and those that did not have the particular GARP response type (“negative”). The significance of the within drug difference for the two group was evaluated using a t-test, and the table lists the associated p-value. Finally, the tables list an efficacy rating (Decreased, Neutral, or Enhanced) for each drug/GARP combination based on whether or not patients with a positive signature displayed significantly inferior, neutral, or superior response respectively. GARP-1 positive subjects experienced enhanced response to olanzapine and risperidone treatment as well as decreased response to perphenazine and ziprasidone (Table 4). GARP-2 positive subjects experienced enhanced response to perphenazine and ziprasidone treatment as well as decreased response to clozapine and quetiapine (Table 5). GARP-3 positive subjects experienced enhanced response to quetiapine treatment as well as decreased response to risperidone (Table 6). GARP-4 positive status did not produce significantly different response in subjects treated with the CATIE drugs, although clozapine did show a numerically better response in GARP-4 positive patients (Table 7).

TABLE 4
Delta PANSS for subjects stratified by GARP-1 status

| | | | | | | | | Relative |
|---|---|---|---|---|---|---|---|---|
| | negative | | | positive | | | | efficacy |
| Treatment | N | Mean | SD | N | Mean | SD | P-value | (positive) |
| Clozapine | 19 | −15.32 | 27.13 | 5 | −14.00 | 13.93 | 0.92 | Neutral |
| Olanzapine | 173 | −7.48 | 14.86 | 47 | −14.83 | 18.73 | 0.005 | Enhanced |
| Perphenazine | 97 | −6.40 | 18.18 | 25 | 2.84 | 15.21 | 0.02 | Decreased |
| Quetiapine | 160 | −0.53 | 16.77 | 47 | −1.87 | 17.77 | .63 | Neutral |
| Risperidone | 183 | −4.55 | 17.75 | 37 | −10.76 | 20.44 | .06 | Enhanced |
| Ziprasidone | 114 | −2.94 | 14.27 | 25 | 12.72 | 19.27 | 0.000008 | Decreased |
| Grand Total | 746 | −4.64 | 16.99 | 186 | −4.65 | 20.40 | 0.99 | Neutral |

TABLE 5
Delta PANSS for subjects stratified by GARP-2 status

| | | | | | | | | Relative |
|---|---|---|---|---|---|---|---|---|
| | negative | | | positive | | | | efficacy |
| Treatment | N | Mean | SD | N | Mean | SD | P-value | (positive) |
| Clozapine | 12 | −24.33 | 23.47 | 12 | −5.75 | 23.15 | 0.06 | Decreased |
| Olanzapine | 110 | −10.73 | 16.29 | 110 | −7.37 | 15.62 | 0.12 | Neutral |
| Perphenazine | 60 | 0.52 | 17.03 | 62 | −9.37 | 17.60 | 0.002 | Enhanced |
| Quetiapine | 111 | −2.86 | 16.99 | 96 | 1.51 | 16.73 | 0.06 | Decreased |
| Risperidone | 104 | −4.65 | 20.77 | 116 | −6.44 | 15.87 | 0.47 | Neutral |
| Ziprasidone | 59 | 3.51 | 19.65 | 80 | −2.80 | 12.93 | 0.02 | Enhanced |
| Grand Total | 456 | −4.46 | 19.06 | 476 | −4.80 | 16.33 | 0.77 | Neutral |

TABLE 6
Delta PANSS for subjects stratified by GARP-3 status

| | negative | | | positive | | | | Relative |
|---|---|---|---|---|---|---|---|---|
| Treatment | N | Mean | SD | N | Mean | SD | P-value | efficacy (positive) |
| Clozapine | 22 | −13.6 | 25.1 | 2 | −31.0 | 17.0 | 0.35 | Neutral |
| Olanzapine | 182 | −9.5 | 16.2 | 37 | −6.3 | 15.2 | 0.26 | Neutral |
| Perphenazine | 103 | −5.5 | 17.9 | 19 | 0.7 | 17.9 | 0.17 | Neutral |
| Quetiapine | 171 | −0.1 | 16.7 | 36 | −4.3 | 18.2 | 0.19 | Neutral |
| Risperidone | 189 | −6.9 | 17.2 | 30 | 2.1 | 23.2 | 0.01 | Decreased |
| Ziprasidone | 123 | −0.1 | 15.9 | 15 | −4.5 | 11.8 | 0.31 | Neutral |
| Grand Total | 790 | −5.0 | 17.4 | 139 | −3.1 | 18.4 | 0.15 | Neutral |

TABLE 7
Delta PANSS for subjects stratified by GARP-4 status

| | negative | | | positive | | | | Relative |
|---|---|---|---|---|---|---|---|---|
| Treatment | N | Mean | SD | N | Mean | SD | P-value | efficacy (positive) |
| Clozapine | 21 | −13.4 | 24.6 | 3 | −26.3 | 27.0 | 0.41 | Neutral |
| Olanzapine | 200 | −9.0 | 16.4 | 20 | −9.3 | 11.2 | 0.94 | Neutral |
| Perphenazine | 110 | −4.7 | 17.6 | 12 | −3.0 | 21.3 | 0.76 | Neutral |
| Quetiapine | 185 | −0.7 | 17.2 | 22 | −1.6 | 15.5 | 0.81 | Neutral |
| Risperidone | 189 | −5.4 | 18.5 | 31 | −6.7 | 17.4 | 0.72 | Neutral |
| Ziprasidone | 124 | 0.4 | 16.5 | 15 | −4.3 | 14.7 | 0.30 | Neutral |
| Grand Total | 829 | −4.5 | 17.8 | 103 | −5.9 | 16.6 | 0.91 | Neutral |

### Example 3

### Clinical Utility of the Determining GARP Genetic Signature Status

General Considerations: Effect Size of GARP Genetic Signature Status in Comparison to those for various antipsychotic drug treatments

The primary rationale for determining the GARP status of a patient is to alter medical practice in some manner. Currently in the psychiatric space, physicians lack useful tools to help them select the most efficacious antipsychotic medication for individual patients. All of the currently approved antipsychotics demonstrated superiority to placebo for treating psychotic symptoms as part of their approval process at the FDA. Examples of comparative effectiveness can be found in any New Drug Approval, e.g. the olanzapine approval package (Zyprexa (olanzapine) Approval Package NDA 20-592. FDA, Center for Drug Evaluation and Research 1996 Available from on the internet at the following site accessdata.fda.gov/drugsatfda_docs/nda/96/020592_Original_Approval_Pkg %20.pdf). However, a recently published meta-analysis of registration quality clinical trials showed only modest improvements over placebo.

Table 8 shows the results for some of the atypical antipsychotics used in the CATIE study. These drugs had effect sizes ranging from −0.42 to −0.59. The effect size is defined as the difference in the mean response for each drug minus the change in the placebo arm, divided by the standard deviation. In this case, a negative number means a greater improvement in psychopathology, i.e. a greater reduction in PANSS. The effect size for drugs with significant GARP type differences are of a similar magnitude, i.e. |0.4| or greater. The effect size is calculated by dividing the difference between the means by the standard deviation of the population. For the CATIE study as a whole this standard deviation is 17.7 PANSS units. Thus, for those groups with significant differences, those differences are of similar magnitude as the difference between the drug and a placebo.

TABLE 8
Comparative Effect Sizes of Atypical Antipsychotics
vs. Placebo and First Generation Antipsychotics

| | Number of | Effect Size | Effect Size |
|---|---|---|---|
| Drug | Studies | vs. Placebo^{a} | vs. FGA^{b} |
| Olanzapine | 6 | −0.59 | −0.21 |
| Quetiapine | 5 | −0.42 | 0.01 |
| Risperidone | 7 | −0.59 | −0.25 |
| Ziprasidone | 4 | −0.48 | −0.04 |

^{a}Leucht et al. (2009)
^{b}FGA = first generation antipsychotics. Results from Davis et al. (2003).

Another recent meta-analysis compared the effectiveness of the various atypical antipsychotics to each other. This study used the weighted mean difference in PANSS score (absolute not percent difference). Olanzapine consistently showed modest superiority to quetiapine, risperidone, and Ziprasidone, in this study (Table 9). The inventors obtained similar results for the CATIE study when patients were not segmented based on GARP genetic signature (CATIE unsegmented, Table 8). The modest increase in efficacy for olanzapine in the unsegmented population is much smaller than the efficacy improvement observed when comparing various GARP status subjects in the CATIE sample as shown in Tables 4, 5, 6, and 7 above.

TABLE 9
Mean Difference Between Olanzapine
and Other Atypical Antipsychotics

| Olanzapine vs. | Meta-analysis^{a} | CATIE unsegemented^{b} |
|---|---|---|
| Risperidone | −1.9 (−3.3 to −0.6) | −3.5 |
| Quetiapine | −3.7 (−5.4 to −1.9) | −6.2 |
| Ziprasidone | −8.3 (−11.0 to −5.6) | −4.9 |

^{a}Leucht Am J Psychiatry 2009; 166: 152-163)
^{b}CATIE phase 1 Caucasian LOCF olanzapine without genetic stratification versus each of the above antipsychotics as determined by the inventors.

A. Using GARP Status to Direct Treatment with Individual Antipsychotics

Comparing efficacy between genetically stratified groups, both in patients of with different versions of a genetic marker (e.g. GARP-1 positive versus negative) treated with the same drug and between patients with the same genetic marker treated with different drugs, has significant clinical utility. The within drug comparison can provide insight has to whether or not the existing clinical expectation for a given drug is likely to for the patient. For example, olanzapine has a clinical profile of superior efficacy compared to the other commonly used antipsychotics (Leucht 2009), and clozapine has a long history of being the drug of last resort for treatment resistant schizophrenia. However, segmentation by GARP status can alter this perceived and general accepted clinical profile. For example, GARP-1 positive patients respond significantly better to olanzapine than GARP-1 negative patients (Table 4). Therefore, GARP-1 positive patients have a better response profile than expect while GARP-1 negative patients have a worse profile than expected. Similarly, GARP-2 positive patients (representing 50%) responded nearly 20 points worse than GARP-2 negative patients to clozapine (Table 5).

All of the antipsychotics have a range of moderate to severe side effects. These include inducing movement disorders, blood disorders, and rapid weight gain with attendant metabolic syndrome. The more efficacious drugs also have the most severe weight gain liability. Thus any treatment decision by a physician must trade off badly needed efficacy with concern over adverse events. For example, olanzapine was once the most commonly prescribed antipsychotic but by 2011 was the fourth best-selling antipsychotic with roughly one-third the market share of the leading antipsychotic quetiapine (IMS).

B.1 Using GARP Status to Determine which Patients should Receive or Avoid Clozapine

GARP-2 positive patients responded poorly to clozapine. Clozapine should be avoided in GARP-2 positive patients and used in GARP-2 negative patients. Clozapine is neither indicated nor contraindicated for patients with GARP-1 and GARP-3 genetic signatures (Table 10).

TABLE 10
Clozapine use

| | negative | | | positive | | | |
|---|---|---|---|---|---|---|---|
| Response type | N | Mean | SD | N | Mean | SD | P-value |
| GARP-2 | 12 | −24.33 | 23.47 | 12 | −5.75 | 23.15 | 0.06 |

B.2 Using GARP Status to Determine which Patients should Receive or Avoid Olanzapine

GARP-1 positive subjects responded particularly well to olanzapine. This efficacy advantage is over and above the advantage usually attributed to olanzapine. Conversely, GARP-1 negative subject did not respond as well as expected. Thus, olanzapine should be used in GARP-1 positive patients and avoided in GARP-1 negative patients (see Table 11).

TABLE 11
Olanzapine use

| | negative | | | positive | | | |
|---|---|---|---|---|---|---|---|
| Response type | N | Mean | SD | N | Mean | SD | P-value |
| GARP-1 | 173 | −7.48 | 14.86 | 47 | −14.83 | 18.73 | 0.005 |

B.3 Using GARP Status to Determine which Patients should Receive or Avoid Quetiapine—

GARP-2 positive subjects responded worse to quetiapine than negative subjects, with the average patient have increased psychopathology scores. Thus, quetiapine should be avoided in GARP-2 positive patients and considered in GARP-2 negative patients (see Table 12).

TABLE 12
Quetiapine use

| | negative | | | positive | | | |
|---|---|---|---|---|---|---|---|
| Response type | N | Mean | SD | N | Mean | SD | P-value |
| GARP-2 | 111 | −2.86 | 16.99 | 96 | 1.51 | 16.73 | 0.06 |

B.4 Using GARP Status to Determine which Patients should Receive or Avoid Perphenazine—

GARP-2 positive patients responded better than GARP-2 negative patients. Thus, perphenazine should be used for GAPR-2 positive patients and avoided for GARP-2 negative patients (Table 13).

TABLE 13
Perphenazine use

| | negative | | | positive | | | |
|---|---|---|---|---|---|---|---|
| Response type | N | Mean | SD | N | Mean | SD | P-value |
| GARP-2 | 60 | 0.52 | 17.03 | 62 | −9.37 | 17.60 | 0.002 |

B.5 Using GARP Status to Determine which Patients should Receive or Avoid Risperidone—

GARP-3 positive patients responded poorly to risperidone, with the average patient getting worse. Therefore, risperidone should be avoided in GARP-3 positive patients and used in GARP-3 negative patients (Table 14).

TABLE 14
Risperidone use

| | negative | | | positive | | | |
|---|---|---|---|---|---|---|---|
| Response type | N | Mean | SD | N | Mean | SD | P-value |
| GARP-3 | 189 | −6.9 | 17.2 | 30 | 2.1 | 23.2 | 0.01 |

B.6 Using GARP Status to Determine which Patients should Receive or Avoid Ziprasidone—

GARP-1 positive patients responded particularly poorly to ziprasidone. Thus, ziprasidone should be avoided for patients that are GARP-1 positive and used in GAP R-1 negative subjects.

TABLE 15
Ziprasidone use

| | |
|---|---|
| negative | positive |

| Response type | N | Mean | SD | N | Mean | SD | P-value |
|---|---|---|---|---|---|---|---|
| GARP-1 | 114 | −2.94 | 14.27 | 25 | 12.72 | 19.27 | 0.000008 |

### C. Using GARP Status to Prioritize Drugs for Treatment

The previous section discusses the comparison of the response to a given drug based on GARP status. This section focuses on selecting a drug from a menu for patients with a given GARP status, i.e. comparing response across drugs. Part of the decision criteria can include the safety profile of the drug.

### C.1 Optimizing Treatment Selection for GARP-1 Positive Patients.

Table 16 shows response to different drugs for patients that are GARP-1 positive. Olanzapine, clozapine, and risperidone demonstrate significant efficacy compared to perphenazine, quetiapine, and ziprasidone (p-value=1×10⁻⁸), with olanzapine and clozapine performing numerically better than risperidone. Clozapine is always challenging due to not only its metabolic side effects but also because of the requirement for frequent blood tests for a serious adverse event. Olanzapine is somewhat more effective than risperidone in the GARP-1 positive group but has a greater weight gain liability. Thus, GARP-1 positive patients should be treated with one of these three drugs: clozapine, olanzapine, or risperidone.

Neither quetiapine nor perphenazine demonstrated efficacy in this group. Ziprasidone treatment led to a worsening of symptoms. Thus, Perphenazine, quetiapine, and ziprasidone should not be used and alternative medications selected for GARP-1 positive patients.

TABLE 16
Response in GARP-1 positive individuals

| Treatment | N | Mean delta PANSS |
|---|---|---|
| Olanzapine | 47 | −14.8 |
| Clozapine | 5 | −14.0 |
| Risperidone | 37 | −10.8 |
| Quetiapine | 47 | −1.9 |
| Perphenazine | 25 | 2.8 |
| Ziprasidone | 25 | 12.7 |

### C.2 Optimizing Treatment Selection for GARP-2 Positive Patients.

As shown in Table 17, for patients that are GARP-2 positive, the group consisting of perphenazine, olanzapine, risperidone and clozapine displayed superior efficacy compared to ziprasidone and quetiapine (p-value=9×10⁻⁶). Perphenazine not only demonstrated the greatest efficacy but GARP-2 positive patients responded significantly better than GARP-2 negative patients. This suggests first line therapy with perphenazine might be warranted. Olanzapine and risperidone displayed similar response, in contrast with the expected superiority of olanzapine. Therefore, risperidone should be selected first due to less weight gain liability in accordance with current practice. Clozapine should be avoided as the risk of serious side effects remain while the expected efficacy advantage does not exist. Additionally, quetiapine should be avoided due to lack of efficacy.

TABLE 17
Response in GARP-2 positive individuals

| Treatment | N | Mean delta PANSS |
|---|---|---|
| Perphenazine | 62 | −9.3 |
| Olanzapine | 110 | −7.4 |
| Risperidone | 116 | −6.4 |
| Clozapine | 12 | −5.8 |
| Ziprasidone | 80 | −2.8 |
| Quetiapine | 96 | 1.5 |

### C.3 Optimizing Treatment Selection for GARP-3 Positive Patients.

As shown in Table 18, for patients that are GARP-3 positive, clozapine provides a clear efficacy advantage to the other drugs (p-value=3×10⁻²). Interestingly, patients in this group experienced significant efficacy but did NOT experience weight gain. GARP-3 positive patients lost on average 4% of their body weight while GARP-3 negative patients gained an average of 6% of their body weight, p-value=0.01. GARP-3 positive patients should NOT receive risperidone or perphenazine. GARP-3 positive patients responded significantly better to quetiapine than GARP-3 negative subjects. Therefore quetiapine might be the next selection after clozapine given quetiapine's lower weight gain liability than olanzapine. Ziprasidone displayed similar efficacy in GARP-3 positive subjects and could be used as a substitute for quetiapine. While olanzapine displayed the second best efficacy, the difference was minor compared to quetiapine and ziprasidone which have superior safety profiles.

TABLE 18
Response in GARP-3 positive individuals

| Treatment | N | Mean delta PANSS |
|---|---|---|
| Clozapine | 2 | −31 |
| Olanzapine | 37 | −6.3 |
| Ziprasidone | 15 | −4.5 |
| Quetiapine | 36 | −4.3 |
| Perphenazine | 19 | 0.7 |
| Risperidone | 30 | 2.1 |

### C.4 Optimizing Treatment Selection for GARP-4 Positive Patients.

As shown in Table 19, for patients that are GARP-3 positive, clozapine provides a clear efficacy advantage to the other drugs (p-value=4×10⁻⁴). Olanzapine displayed the second best response in GARP-4 positive patients and should be considered for use, followed by risperidone.

TABLE 19
Response in GARP-4 positive individuals

| Treatment | N | Mean delta PANSS |
|---|---|---|
| Clozapine | 3 | −26.3 |
| Olanzapine | 20 | −9.3 |
| Risperidone | 31 | −6.7 |
| Ziprasidone | 15 | −4.3 |
| Perphenazine | 12 | −3.0 |
| Quetiapine | 22 | −1.6 |

### Example 5

### Classification of Drugs by Weight Gain Liability and its Interaction with GARP Status

A. GARP status impact on response in relation to weight gain liability of drugs

Historically, antipsychotic drugs are known for their weight gain liability. Indeed, almost all of the drugs induce weight gain in treatment naïve patients. Even within that context, a hierarchy has emerged with clozapine and olanzapine classified as high risk and risperidone and quetiapine as medium risk for weight gain. Other drugs, including ziprasidone and aripiprazole, are classified as low risk. (Haynes Curr Psychiatry Rev. 2012 February 1; 8(1): 25-36) These classifications were confirmed by inventors in an analysis of the CATIE data that yielded similar results, with the high, medium, and low risk drugs inducing 4.4%, 1.4% and −0.9% weight gain respectively. These differences were highly significant when tested using Analysis of Variance, ANOVA, p-value=1×10⁻¹⁰

GARP-1 status predicts response to the drugs with different weight gain classifications. Specifically as shown in Table 19, GARP-1 positive status correlates with NON-response to the low weight-gain medications aripiprazole and ziprasidone. Thus, for GARP-1 patients, low weight gain drugs should not be used. Conversely, medium and high weight gain drugs demonstrated superior response in GARP-1 positive patients and should be used for those patients, with the two drugs, olanzapine and clozapine, with highest weight gain liability providing the greatest efficacy. The poor response of low weight gain drugs in GARP-1 positive patients also holds true for aripiprazole in the if the phase 3 data of the CATIE study were to be included (aripiprazole was only prescribed in the non-blinded phase 3).

TABLE 19
Antipsychotic weight gain risk, low versus medium and high, impacts
response based on GARP-1 status.

| Weight | GAPR-1 negative | | | GARP-1 positive | | | |
|---|---|---|---|---|---|---|---|
| Gain risk | N | Mean | SD | N | Mean | SD | p-value |
| Low | 211 | −4.5 | 16.2 | 50 | 7.8 | 17.9 | 0.000004 |
| Medium | 535 | −4.7 | 17.3 | 136 | −9.2 | 19.4 | 0.007 |
| and High | | | | | | | |

B. Drug Device Combinations for GLP-1 Analogues and GLP1R Agonists for Patients with GARP Status that Recommends a High Risk Weight Gain Drug, Olanzapine or Clozapine.

In Phase 1 of CATIE, 304 of the 622 subjects (49%) included in analysis of efficacy data had GARP-1, GARP-3, or GARP-4 status. GARP-1 positive status indicates that the best efficacy is achieved using a high weight gain drug, olanzapine or clozapine, or medium weight gain drug, risperidone and quetiapine. Similarly, the high weight gain drug clozapine displayed the best efficacy in GARP-3 and GARP-4 positive patients.

GLP1R is the glucogon-like peptide 1 (GLP1) receptor. GLP1R is a drug target for diabetes and weight loss medications. Specifically, GLP1R agonists like exenatide and liraglutide have been approved by the FDA to treat type 2 diabetes. Both drugs increase insulin secretion and also cause weight loss. Interestingly, the GLP1R-2 haplotype contains a coding variant that has been shown to have lower insulin release in response to GLP1R agonist therapy. Presence of at least one copy of this haplotype defines GARP-2 positive status. GARP-2 positive patients display superior response to low weight gain risk drugs compared to GARP-2 negative patients, p-value=3×10⁻⁴. Conversely, GARP-2 positive patients display lower response to the high weight gain risk drugs, olanzapine and clozapine, compared to GARP-2 negative subjects, p-value=0.03.

Thus the drug device combination consists of a genetic test to determine GARP status. If the patient is GARP-1 positive, then the patient should be prescribed olanzapine as first line therapy. Additionally, a GLP1R agonist should also be concurrently prescribed in order to mitigate the risk of developing metabolic side-effects including weight gain and metabolic syndrome. In some embodiments the olanzapine and the GLP1R agonist are long lasting injectable formulation. In some embodiments, both drugs are administered in a single injection. Second line therapy for GARP-1 positive subjects should be either risperidone or clozapine; risperidone should be preferred due to the lack of required blood monitoring. With either clozapine or olanzapine, GARP-1 positive status indicates a need for co-administration of a GLP1R agonist. Risperidone and quetiapine may also require coadministration of a GLP1R agonist to offset metabolic side-effects. Low weight gain drugs should be avoided. Similarly, for GARP-3 and GARP-4 positive patients, clozapine should be used, and a GLP1R agonist should be administered. For GARP-2 positive individuals, a low weight gain risk drug should be utilized as first line therapy.

Thus, a drug-device combination comprises determining GARP status with a genetic test, administering an antipsychotic based on GARP status (low weight gain drug for GARP-2 positive patients or high weight gain drug for GARP-1 and GARP-3 positive patients), and administering a GLP1R agonist if a high weight gain drug is selected.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 5,143,854
U.S. Pat. No. 5,272,071
U.S. Pat. No. 5,288,514
U.S. Pat. No. 5,288,644
U.S. Pat. No. 5,384,261
U.S. Pat. No. 5,424,186
U.S. Pat. No. 5,451,683
U.S. Pat. No. 5,491,224
U.S. Pat. No. 5,510,270
U.S. Pat. No. 5,527,681
U.S. Pat. No. 5,776,688
U.S. Pat. No. 5,800,998
U.S. Pat. No. 5,837,832
U.S. Pat. No. 5,858,659
U.S. Patent Publication No. 2003/0108938
U.S. Patent Publication No. 2004/0014095
U.S. Patent Publication No. 2006/0177851
U.S. Patent Publication No. 2009/0012371
PCT US/93/04145
WO 91/06667
WO 92/10092
WO 95/11995
WO 98/20019
WO 99/57318
WO 2009/092032
WO 2009/089120

WO 2009/082743
Albertson et al., Breast Cancer Research and Treatment 78:289-298 (2003).
Alderborn et al., Genome Research 10(8):1249-1258 (2000).
Ausubel et al., Current Protocols in Molecular Biology, John Wiley & Sons, N.Y. (2003).
Badner et al., Mol. Psychiatry. 7:405-411 (2002).
Bao et al., PolymiRTS Database: linking polymorphisms in microRNA target sites with complex traits. Nucleic Acids Res. 35(Database issue), D51-D54 (2007).
Barrett et al., Haploview: analysis and visualization of LD and haplotype maps. Bioinformatics. 21(2), 263-265 (2005).
Barrett, Haploview: Visualization and analysis of SNP genotype data. Cold Spring Harb. Protoc. 2009(10), db-(2009).
Chen et al., Genome Research 9(5):492-498 (1999).
Church and Gilbert, Proc. Natl. Acad. Sci. USA 81:1991-1995 (1988).
Cooper-Casey et al., Mol. Psychiatry. 10:651-656 (2005).
Cotton et al., Proc. Natl. Acad. Sci. USA 85:4397-4401 (1985).
Devlin et al., Mol. Psychiatry. 7:689-694 (2002).
Eckert et al., PCR Methods and Applications 1:17 (1991).
Eichelbaum et al., Clin. Exp. Pharmacol. Physiol. 23:983-985 (1996).
Fallin et al., Am. J. Hum. Genet. 77:918-936 (2005).
Fallin et al., Am. J. Hum. Genet. 73:601-611 (2003).
Flavell et al., Cell 15:25 (1978).
Geever et al., Proc. Natl. Acad. Sci. USA 78:5081 (1981).
Guatelli et al., Proc. Nat. Acad. Sci. USA 87:1874 (1990).
Guttmacher and Collins JAMA 294:1399-402 (2005).
Hamer and Simpson, Last observation carried forward versus mixed models in the analysis of psychiatric clinical trials. Am. J. Psychiatry 166(6), 639-641 (2009).
Jablensky, Mol. Psychiatry. 11: 815-836 (2006).
Kane, Pharmacologic treatment of schizophrenia. Biol. Psychiatry 46(10), 1396-1408 (1999).
Kay et al., Schizophr. Bull. 13:261-276 (1987).
Kay et al., Br. J. Psychiatry Suppl:59-67 (1989).
Kirov et al., J. Clin. Invest. 115:1440-1448 (2005).
Klein et al., Proc. Natl. Acad. Sci. USA 96:4494-4499 (1999).
Kwoh et al., Proc. Natl. Acad. Sci. USA 86:1173 (1989).
Landegren et al., Science 241:1077 (1988).
Leucht et al., Schizophr. Res. 79:231-238 (2005).
Leucht et al., Am J Psychiatry, 166:152-163 (2009).
Lieberman et al., Effectiveness of antipsychotic drugs in patients with chronic schizophrenia. N. Engl. J. Med. 353(12), 1209-1223 (2005).
Linder et al., Clin. Chem. 43:254-266 (1997).
Liu et al., Pharmacogenomics. 13:1227-1237 (2012).
Maniatis et al., Proc. Natl. Acad. Sci. USA 99:2228-2233 (2002).
Manolio et al., New models of collaboration in genome-wide association studies: the Genetic Association Information Network. Nat. Genet. 39(9), 1045-1051 (2007).
Mattila et al., Nucleic Acids Res. 19:4967 (1991).
McClay et al., Genome-wide pharmacogenomic analysis of response to treatment with antipsychotics. Mol. Psychiatry 16(1), 76-85 (2011).
McPherson et al., PCR Basics: From Background to Bench, Springer-Verlag, (2000).
Meltzer and Huang, In vivo actions of atypical antipsychotic drug on serotonergic and dopaminergic systems. Prog. Brain Res. 172, 177-197 (2008).
Morton et al., Proc. Natl. Acad. Sci. USA 98(9):5217-21 (2001).
Myers et al., Science 230:1242 (1985).
Nath and Johnson, Biotechnic. Histochem. 73(1):6-22 (1998).
Need et al., Pharmacogenetics of antipsychotic response in the CATIE trial: a candidate gene analysis. Eur. J. Hum. Genet. 17(7), 946-957 (2009a).
Need et al., A genome-wide investigation of SNPs and CNVs in schizophrenia. PLoS. Genet. 5(2), e1000373-(2009b).
Nielsen et al., Bioconjugate Chemistry, The American Chemical Society, 5:1 (1994).
Norton et al., Curr. Opin. Psychiatry. 19:158-164 (2006).
Orita et al., Proc. Natl. Acad. Sci. USA 86:2766-2770 (1989).
Owen et al., Mol. Psychiatry. 9:14-27 (2004).
PCR (eds. McPherson et al., IRL Press, Oxford).
PCR Primer: A Laboratory Manual, Dieffenbach and Dveksler, (Eds.).
Practice Guideline for the Treatment of Patients With Schizophrenia American Psychiatric Association, Second Edition, American Psychiatric Association (2004).
Prince et al., Genome Res. 11:152-162 (2001).
Purcell et al., PLINK: a tool set for whole-genome association and population-based linkage analyses. Am. J. Hum. Genet. 81(3), 559-575 (2007).
Raca et al., Genet Test 8(4):387-94 (2004).
Redon et al., Nature 444(7118):444-54 (2006).
Saiki et al., Nature 324:163-166 (1986).
Sanger et al., Proc. Natl. Acad. Sci. USA 74:5463-5467 (1977).
Schafer et al., Nat. Biotechnol. 15:33-39 (1995).
Sheffield et al., Proc. Natl. Acad. Sci. USA 86:232-236 (1989).
Snijders et al., Nat. Genetics 29:263-264 (2001).
Snijders et al., “BAC microarray based comparative genomic hybridization,” in: Zhao et al. (eds), Bacterial Artificial Chromosomes: Methods and Protocols, Methods in Molecular Biology, Humana Press (2002).
Stone et al., Nature 455(7210):237-41 (2008).
Stoneking et al., Am. J. Hum. Genet. 48:370-382 (1991).
Stroup et al., The National Institute of Mental Health Clinical Antipsychotic Trials of Intervention Effectiveness (CATIE) project: schizophrenia trial design and protocol development. Schizophr. Bull. 29(1), 15-31 (2003).
Sullivan et al., Genomewide association for schizophrenia in the CATIE study: results of stage 1. Mol. Psychiatry 13(6), 570-584 (2008).
Tapper et al., Proc. Natl. Acad. Sci. USA 102(33):11835-11839 (2005).
The International HapMap Consortium, Nature 426:789-796 (2003).
The International HapMap Consortium, Nature 437:1299-1320 (2005).
Underhill et al., Genome Res. 7:996-1005 (1997).
van den Oord et al., A systematic method for estimating individual responses to treatment with antipsychotics in CATIE. Schizophr. Res. 107(1), 13-21 (2009).
Walsh et al., Science 320(5875):539-43 (2008).
Wang et al., Pharmacogenetics 13:555-64 (2003).
Weinshilboum and Wang, Nature Rev. Drug Discovery 3:739-748 (2004).
Weinshilboum and Wang, Annu. Rev. Genomics Hum. Genet. 7:223-45 (2006).
Wheeless et al., Cytometry 17:319-326 (1994).
Williams et al., Hum. Mol. Genet. 8:1729-1739 (1999).
Wu and Wallace, Genomics 4:560 (1989).
Ziebarth et al., PolymiRTS Database 2.0: linking polymorphisms in microRNA target sites with human diseases and complex traits. Nucleic Acids Res. 40(Database issue), D216-D221 (2012).

What is claimed is:

1. A method of treating a subject with an antipsychotic medication comprising:
obtaining genetic information from the subject comprising the sequence of SNPs rs6923761, rs2300615, and rs1042044 to provide the GLP1R Antipsychotic Response Phenotype (GARP) genetic signature of the subject; and
administering an antipsychotic medication to the subject based on the GARP genetic signature of the subject.

2. The method of claim 1, wherein obtaining the sequence of the SNPs comprises directly determining the sequence at SNP positions rs6923761, rs2300615, and rs1042044.

3. The method of claim 1, wherein obtaining the sequence of the SNPs comprises determining the sequence at SNPs in linkage disequilibrium with SNP positions rs6923761, rs2300615, and rs1042044.

4. The method of claim 3, wherein the obtaining the sequence of the SNPs comprises determine the sequence at SNP positions rs9296283 rs7766275 rs2300615 and rs1042044.

5. The method of claim 3, wherein the obtaining the sequence of the SNPs comprises determine the sequence at SNP positions rs10305439 rs742764 rs2268650 rs910170 rs6923761 rs9296283 rs7766275 rs2300615 rs2235868 and rs1042044.

6. The method of any one claims 1-5 wherein, obtaining the sequence of the SNPs comprises obtaining a sample from the subject and determining the genetic sequences in the sample.

7. The method of claim 1, further comprising selecting a subject having a GARP-1 genetic signature corresponding to SNPs rs6923761 (G), rs2300615 (T), and rs1042044 (A) on both chromosomes and administering first antipsychotic medication selected from the group consisting of olanzapine, risperidone, and clozapine to the selected subject.

8. The method of claim 7, further comprising:
determining the efficacy or the side effects associated with administering the first antipsychotic medication.

9. The method of claim 8, further comprising:
administering a second antipsychotic medication to the subject, selected from the group consisting of olanzapine, risperidone, and clozapine, if the first antipsychotic medication is determined to have insufficient efficacy or unacceptable side effects.

10. The method of claim 7, wherein the first antipsychotic medication is olanzapine:

11. The method of claim 10, wherein the subject is treated with a GLP1R agonist in conjunction with olanzapine.

12. The method of claim 7, wherein the first antipsychotic medication is clozapine:

13. The method of claim 12, wherein the subject is treated with a GLP1R agonist in conjunction with clozapine.

14. The method of claim 7, wherein the subject is not administered ziprasidone, perphenazine or aripiprazole.

15. The method of claim 1, further comprising selecting a subject having a GARP-1 genetic signature corresponding to SNPs rs6923761 (G), rs2300615 (T), and rs1042044 (A) on both chromosomes and administering an antipsychotic medication wherein the antipsychotic medication is not ziprasidone, perphenazine or aripiprazole.

16. The method of claim 1, further defined as a method of using the antipsychotic aripiprazole comprising determining whether a subject has the GARP-1 genetic signature corresponding to SNPs rs6923761 (G), rs2300615 (T), and rs1042044 (A) on both chromosomes; and treating the subject with aripiprazole if the subject is GARP-1 negative or treating the subject with a non-aripiprazole antipsychotic if the subject is GARP-1 positive.

17. The method of claim 1, further defined as a method of using olanzapine, the method comprising determining whether a subject has the GARP-1 genetic signature corresponding to SNPs rs6923761 (G), rs2300615 (T), and rs1042044 (A) on both chromosomes; and treating the subject with olanzapine if the subject is GARP-1 positive or treating the subject with a non-olanzapine antipsychotic if the subject is GARP-1 negative.

18. The method of claim 1, further defined as a method of using the antipsychotic ziprasidone, the method comprising determining whether a subject has the GARP-1 genetic signature corresponding to SNPs rs6923761 (G), rs2300615 (T), and rs1042044 (A) on both chromosomes; and
treating the subject with ziprasidone if the subject is GARP-1 negative or treating the subject with a non-ziprasidone antipsychotic if the subject is GARP-1 positive.

19. The method of claim 1, further comprising selecting a subject having a GARP-2 genetic signature corresponding to:
(a) SNPs rs6923761 (A), rs2300615 (T), and rs1042044 (C) on one chromosome; and
(b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and rs1042044 (C); or (iv) SNPs rs6923761 (G), rs2300615 (T), and rs1042044 (C) on a second chromosome; and
administering a first antipsychotic medication selected from the group consisting of perphenazine, risperidone and ziprasidone to the selected subject.

20. The method of claim 19, further comprising:
determining the efficacy or the side effects associated with administering the first antipsychotic medication.

21. The method of claim 20, further comprising:
administering a second antipsychotic medication to the subject, selected from the group consisting of perphenazine, risperidone and ziprasidone, if the first antipsychotic medication is determined to have insufficient efficacy or unacceptable side effects.

22. The method of claim 19, wherein the subject is not administered clozapine, olanzapine, or quetiapine.

23. The method of claim 1, further comprising selecting a subject having a GARP-2 genetic signature corresponding to:
(a) SNPs rs6923761 (A), rs2300615 (T), and rs1042044 (C) on one chromosome; and
(b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and
administering an antipsychotic medication to the subject, wherein the antipsychotic medication is not clozapine, olanzapine, or quetiapine.

24. The method of claim 1, further defined as a method of using the antipsychotic clozapine, the method comprising determining whether a subject has the GARP-2 genetic signature corresponding to:
(a) SNPs rs6923761 (A), rs2300615 (T), and rs1042044 (C) on one chromosome; and
(b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and
treating the subject with clozapine if the subject is GARP-2 negative or treating the subject with a non-clozapine antipsychotic if the subject is GARP-2 positive.

25. The method of claim 1, further defined as a method of using the antipsychotic perphenazine, the method comprising determining whether a subject has the GARP-2 genetic signature corresponding to:
(a) SNPs rs6923761 (A), rs2300615 (T), and rs1042044 (C) on one chromosome; and
(b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and
treating the subject with perphenazine if the subject is GARP-2 positive or treating the subject with a non-perphenazine antipsychotic if the subject is GARP-2 negative.

26. The method of claim 1, further defined as a method of using the antipsychotic quetiapine, the method comprising determining whether a subject has the GARP-2 genetic signature corresponding to:
(a) SNPs rs6923761 (A), rs2300615 (T), and rs1042044 (C) on one chromosome; and
(b) (i) SNPs rs6923761 (G), rs2300615 (T), and rs1042044 (A); (ii) SNPs rs6923761 (A), rs2300615 (T), and rs1042044 (C); (iii) SNPs rs6923761 (G), rs2300615 (G), and (iv) rs1042044 (C) or SNPs rs6923761 (G), rs2300615 (T), and rs1042044 (C) on a second chromosome; and
treating the subject with quetiapine if the subject is GARP-2 negative or treating the subject with a non-quetiapine antipsychotic if the subject is GARP-2 positive.

27. The method of claim 1, further comprising selecting a subject having a GARP-3 genetic signature corresponding to:
(a) SNPs rs6923761 (G), rs2300615 (G), and rs1042044 (C) on one chromosome; and
(b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and
administering first antipsychotic medication to the subject selected from the group consisting of clozapine, quetiapine and ziprasidone to the selected subject.

28. The method of claim 27, further comprising:
determining the efficacy or the side effects associated with administering the first antipsychotic medication.

29. The method of claim 28, further comprising:
administering a second antipsychotic medication to the subject, selected from the group consisting of clozapine, quetiapine and ziprasidone, if the first antipsychotic medication is determined to have insufficient efficacy or unacceptable side effects.

30. The method of claim 27, wherein the subject is not administered olanzapine or risperidone.

31. The method of claim 1, further comprising selecting a subject having a GARP-3 genetic signature corresponding to:
(a) SNPs rs6923761 (G), rs2300615 (G), and rs1042044 (C) on one chromosome; and
(b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and
administering an antipsychotic medication to the subject, wherein the antipsychotic medication is not olanzapine or risperidone.

32. The method of claim 1, further defined as a method of using the antipsychotic risperidone, the method comprising determining whether a subject has the GARP-3 genetic signature corresponding to:
(a) SNPs rs6923761 (G), rs2300615 (G), and rs1042044 (C) on one chromosome; and
(b) (i) SNPs rs6923761 (G), rs2300615 (T), and rs1042044 (A); (ii) SNPs rs6923761 (G), rs2300615 (G), and rs1042044 (C) or (iii) SNPs rs6923761 (G), rs2300615 (T), and rs1042044 (C) on a second chromosome; and
treating the subject with risperidone if the subject is GARP-3 negative or treating the subject with a non-risperidone antipsychotic if the subject is GARP-3 positive.

33. The method of claim 1, further comprising selecting a subject having a GARP-1 genetic signature corresponding to SNPs rs6923761 (G), rs2300615 (T), and rs1042044 (A) on both chromosomes; and
administering (i) a first antipsychotic medication selected from the group consisting of olanzapine and clozapine; and (ii) a GLP1R agonist to the selected subject.

34. The method of claim 33, wherein the first antipsychotic medication is olanzapine:

35. The method of claim 33, wherein the GLP1R agonist and the first antipsychotic medication are administered separately.

36. The method of claim 33, further comprising administering a composition comprising a GLP1R agonist and first antipsychotic medication to the subject.

37. The method of claim 36, wherein the composition is administered orally or by injection.

38. The method of any one of claims 1-37, wherein the subject is Caucasian or comprises European ancestry.

39. The method of any one of claims 1-37, wherein the subject is African American or comprises African ancestry.
